(19) United States
(12) Patent Application Publication
(10) Pub. No.: US 2026/0097124 A1
(43) Pub. Date: Apr. 9, 2026

(54) METHOD FOR THE PRODUCTION OF LARGE-RING CYCLODEXTRINS

(71) Applicant: Danmarks Tekniske Universitet, Kongens Lyngby (DK)

(72) Inventors: Sophie Rachel Beeren, Kongens Lyngby (DK); Kasper Hornstrup Hansen, Kongens Lyngby (DK); Andreas Erichsen, Kongens Lyngby (DK); Dennis Larsen, Kongens Lyngby (DK)

(21) Appl. No.: 19/105,169

(22) PCT Filed: Aug. 25, 2023
(86) PCT No.: PCT/EP2023/073406

§ 371 Date: Feb. 20, 2025

(30) Foreign Application Priority Data
Aug. 26, 2022 (EP) 22192440.0

(51) Int. Cl. A61K 47/40; A61K 47/02; A61K 47/26; C08B 30/18

(52) U.S. Cl. CPC A61K 47/40; A61K 47/02; A61K 47/26; C08B 30/18

(57) ABSTRACT

The disclosure relates to a synthetic method of producing delta-cyclodextrin. The method is scalable, and the isolation of the product does not require chromatography.

### TECHNICAL FIELD

The present invention relates to production of macrocyclic organic compounds, specifically cyclodextrins.

### BACKGROUND

Cyclodextrins (CDs) are a family of cyclic oligosaccharides, consisting of a macrocyclic ring of glucopyranose subunits joined by alpha-1,4 glycosidic bonds.

Cyclodextrins are used for the improvement of water-solubility and bioavailability of drugs. Because of the diverse types of application of cyclodextrins, several types of medicinal products may contain cyclodextrins. They are used for example in tablets, aqueous parenteral solutions, nasal sprays and eye drop solutions. Examples of the use of cyclodextrins in medicines on the European market are beta-CD in cetirizine tablets and cisapride suppositories, gamma-CD in minoxidil solution, and examples of the use of beta-cyclodextrin derivatives are SBE-beta-CD in the intravenous antimycotic voriconazole, HP-beta-CD in the antifungal itraconazole, intravenous and oral solutions, and RM-beta-CD in a nasal spray for hormone replacement therapy by 17beta-estradiol. In Germany and Japan there are infusion products on the market, containing alprostadil (prostaglandin E1, PGE1) with alpha-CD (EMA, 2017).

Cyclodextrins typically contain 6 to 8 glucose subunits, termed alpha-cyclodextrin, beta-cyclodextrin, and gamma-cyclodextrin (also abbreviated CD6, CD7, and CD8). Cyclodextrins are commercially produced via enzymatic breakdown of starch or amylose.

New methods to produce cyclodextrins larger than the conventional alpha, beta, and gamma-CD, in high yield and of high purity, without the need for column chromatography, are highly desirable. The provision of such methods could potentially enable large-ring CDs to become commercially available, preferably at a low cost, which in turn would allow for such CDs to be employed as excipients in new drug formulations. There is also potential for large ring CDs to find application in cosmetics or nutrition.

### SUMMARY

The present disclosure provides for methods for producing delta-cyclodextrin and derivatives thereof in good yields and high selectivity, and thus allowing for their isolation without the need for chromatography.

One aspect of the present disclosure provides for a method for producing delta-cyclodextrin or a delta-cyclodextrin derivative, said method comprising mixing:
a. a glucose-based compound,
b. an enzyme such as cyclodextrin glucanotransferase,
c. an ion of formula (B₁₂ClₙH₁₂₋ₙ)²⁻, wherein n is 1 to 12, and
d. a solvent
to form a mixture, and
allowing the mixture to incubate,
thereby obtaining the delta-cyclodextrin or a delta-cyclodextrin derivative.

In one aspect, the delta-cyclodextrin or the delta-cyclodextrin derivative has the structure of formula I:

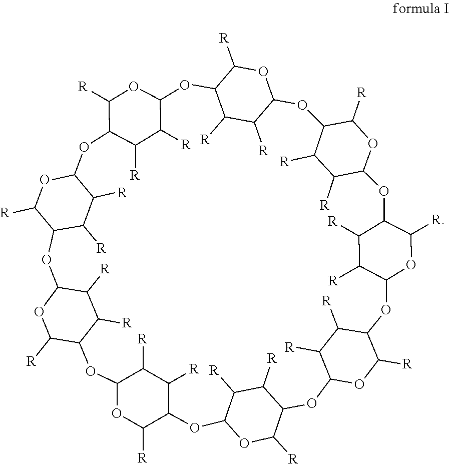

In one embodiment, the delta-cyclodextrin or the delta-cyclodextrin derivative has the structure of formula I, or a salt thereof, wherein each R is independently selected from the group consisting of CH₂OR^{a}, CH₃, CH₂F, CH₂Cl, CH₂Br, CH₂I, CH₂N₃, CH₂CN, CH₂Ra, CH₂N(R^{a})₂, CH₂SR^{a}, CH₂Si(R^{a})₃, CH₂CHO, CH₂COOH, CH₂COOR^{a}, CH₂CON(R^{a})₂, CH₂CHNR^{a}, OR^{a}, H, F, Cl, Br, I, N₃, CN, R^{a}, N(R^{a})₂, SR^{a}, Si(R^{a})₃, CHO, COOH, COOR^{a}, CON(R^{a})₂, and CHNR^{a}, and wherein each R^{a} is independently selected from the group consisting of H, alkyl, substituted alkyl, alkenyl, substituted alkenyl, alkynyl, substituted alkynyl, acyl, substituted acyl, aryl, substituted aryl, benzyl, and substituted benzyl.

In one aspect of the present disclosure, the glucose-based compound has the structure of formula II:

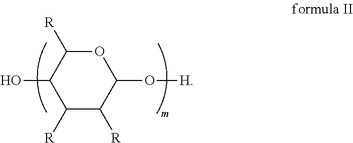

In one embodiment of the present disclosure, the glucose-based compound has the structure of formula II,
wherein each R is independently selected from the group consisting of CH₂OR^{a}, CH₃, CH₂F, CH₂Cl, CH₂Br, CH₂I, CH₂N₃, CH₂CN, CH₂R^{a}, CH₂N(R^{a})₂, CH₂SR^{a}, CH₂Si(R^{a})₃, CH₂CHO, CH₂COOH, CH₂COOR^{a}, CH₂CON(R^{a})₂, CH₂CHNR^{a}, OR^{a}, H, F, C, Br, I, N₃, CN, R^{a}, N(R^{a})₂, SR^{a}, Si(R^{a})₃, CHO, COOH, COOR^{a}, CON(R^{a})₂, and CHNR^{a},
wherein each R^{a} is independently selected from the group consisting of H, alkyl, substituted alkyl, alkenyl, substituted alkenyl, alkynyl, substituted alkynyl, acyl, substituted acyl, aryl, substituted aryl, benzyl, and substituted benzyl, and wherein m is an integer 2 or more.

One aspect of the present disclosure provides for an ion of formula (B₁₂ClₙH₁₂₋ₙ)²⁻. In one embodiment, n is 1 to 12. In one embodiment, n is 1, 2, 3, 4, 5, 6, 7, 8, 9, 10, 11, or 12. In one embodiment, n is 9, 10, 11, or 12. In one embodiment, the ion of formula [B₁₂ClₙH₁₂₋ₙ]²⁻ is selected from the group consisting of (B₁₂Cl₁₂)²⁻, (B₁₂Cl₁₁H)²⁻, (B₁₂Cl₁₀H₂)²⁻, (B₁₂Cl₉H₃)²⁻, (B₁₂Cl₈H₄)²⁻, (B₁₂Cl₇H₅)²⁻, (B₁₂Cl₆H₆)²⁻, (B₁₂Cl₅H₇)²⁻, (B₁₂Cl₄H₃)²⁻, (B₁₂Cl₃H₉)²⁻, (B₁₂Cl₂H₁₀)²⁻, and (B₁₂ClH₁₁)²⁻. In one embodiment, the ion of formula [B₁₂ClₙH₁₂₋ₙ]²⁻ is selected from the group consisting of (B₁₂Cl₁₂)²⁻, (B₁₂Cl₁₁H)²⁻, (B₁₂Cl₁₀H₂)²⁻, and (B₁₂Cl₉H₃)²⁻. In one embodiment, the ion of formula (B₁₂ClₙH₁₂₋ₙ)²⁻ is (B₁₂Cl₁₂)²⁻.

In one aspect of the present disclosure, the enzyme such as the cyclodextrin glucanotransferase is a cyclodextrin glucanotransferase as defined by Enzyme Commission (EC) number 2.4.1.19.

In one specific aspect, the method comprises the steps carried out in the order:
a. mixing:
   i. a glucose-based compound,
   ii. a cyclodextrin glucanotransferase,
   iii. an ion of formula (B₁₂ClₙH₁₂₋ₙ)²⁻, wherein n is 1 to 12, and
   iv. a solvent
   to form a mixture;
b. incubating the mixture by heating;
c. partially evaporating the solvent from mixture to form a solid phase and a liquid phase;
d. separating the solid phase and the liquid phase of the mixture;
e. adding antisolvent to said liquid phase to precipitate the delta-cyclodextrin or the delta-cyclodextrin derivative; and
f. filtering the precipitated delta-cyclodextrin or the delta-cyclodextrin derivative.

The template ion employed in the method of the disclosure can be recovered and reused to carry out the method of the disclosure again. Thus, in one aspect, the method further comprises recovering the ion of formula (B₁₂ClₙH₁₂₋ₙ)²⁻, said recovery comprising:
a. obtaining the supernatant from the precipitation step of the delta-cyclodextrin or the delta-cyclodextrin derivative,
b. evaporating antisolvent under reduced pressure,
c. adding hydrochloric acid, such as 37% hydrochloric acid to obtain a pH between 1 and 4, such as between 1.5 and 2.5,
d. adding a base, such as an amine base, such as an alkyl amine base, such as a trialkyl amine compound, such as triethylamine to precipitate the ion of formula (B₁₂ClₙH₁₂₋ₙ)²⁻ as a salt of said base, and
e. filtering the precipitate,
thereby obtaining the ion of formula (B₁₂ClₙH₁₂₋ₙ)²⁻ as a salt of said base.

One aspect of the present disclosure provides for a delta-cyclodextrin or a delta-cyclodextrin derivative obtained from the method as disclosed herein.

One aspect of the disclosure provides for a composition comprising:
a. a glucose-based compound as disclosed herein,
b. an enzyme such as a cyclodextrin glucanotransferase as disclosed herein, and
c. an ion of formula (B₁₂ClₙH₁₂₋ₙ)²⁻ as disclosed herein.

One aspect of the present disclosure provides for a kit of parts comprising:
a. a glucose-based compound as disclosed herein,
b. a enzyme such as a cyclodextrin glucanotransferase as disclosed herein, and
c. a salt comprising an ion of formula (B₁₂ClₙH₁₂₋ₙ)²⁻ as disclosed herein.

### DESCRIPTION OF DRAWINGS

FIG. 1: HPLC chromatograms showing the conversion of CD6 to a mixture of CD6, CD7 and CD8 when CD6 is treated with CGTase in the absence of a template.

FIG. 2: Distribution of CDs and linear alpha-1,4-glucans (G1-G4) produced overtime when CD6 is treated with CGTase in the absence of template.

FIG. 3: HPLC chromatograms showing the conversion of CD6 to mainly CD9 when CD6 is treated with CGTase in the presence of Na₂B₁₂Cl₁₂.

FIG. 4: Distribution of CDs and linear alpha-1,4-glucans produced over time when CD6 is treated with CGTase in the presence of 4 mM Na₂B₁₂Cl₁₂ at 25° C. First figure: distribution of different CDs formed. Second figure: concentration of all glucan products formed.

FIG. 5: Distribution of CDs and linear alpha-1,4-glucans produced over time when CD6 is treated with CGTase in the presence of 4 mM Na₂B₁₂Cl₁₂ at 5° C. First figure: distribution of different CDs formed. Second figure: concentration of all glucan products formed.

FIG. 6: Distribution of CDs and linear alpha-1,4-glucans produced over time when CD6 is treated with CGTase in the presence of 4 mM Na₂B₁₂Cl₁₂ at 40° C. First figure: distribution of different CDs formed. Second figure: concentration of all glucan products formed.

FIG. 7: Distribution of CDs and linear alpha-1,4-glucans produced over time when CD6 is treated with CGTase in the presence of 2 mM Na₂B₁₂Cl₁₂ at 25° C. First figure: distribution of different CDs formed. Second figure: concentration of all glucan products formed.

FIG. 8: Distribution of CDs produced over time when CD6 is treated with CGTase in the presence of 30 mM Na₂B₁₂Cl₁₂ at 25° C. First figure: distribution of different CDs formed. Second figure: concentration of all glucan products formed.

FIG. 9: HPLC-ESLD chromatogram demonstrating the purity of isolated CD9.

FIG. 10: ¹H NMR spectrum of isolated CD9.

FIG. 11: ¹H NMR spectra of Na₂B₁₂Cl₁₂ in D₂O before (first spectrum) and after (second spectrum) recovery from an enzymatic CD9 production. Note that no peaks are expected, and indeed only the solvent peak is observed in both cases.

FIG. 12: ¹¹B NMR spectra of Na₂B₁₂Cl₁₂ in D₂O before (first spectrum) and after (second spectrum) recovery from an enzymatic CD9 production.

FIG. 13: Mass spectra (MALDI-TOF, positive mode) of Na₂B₁₂Cl₁₂ before (first spectrum) and after (second spectrum) recovery from an enzymatic CD9 production. The major peak corresponds to the sodium adduct. “60k” means “60000” etc.

FIG. 14: MALDI-TOF-MS of reaction mixture after 20 days of treatment of mono-6-deoxy-CD6 in presence of Na₂B₁₂Cl₁₂ in phosphate buffered water at room temperature.

FIG. 15: HPLC-ELS chromatogram of reaction mixture after 20 days of treatment of mono-6-deoxy-CD6 in presence of Na₂B₁₂Cl₁₂ in phosphate buffered water at room temperature.

FIG. 16: Chromatograms (ELSD) showing distribution of linear and cyclic alpha-1,4-glucans in the reaction where starch was treated with CGTase in the presence of Na₂B₁₂Cl₁₂.

FIG. 17: Mass spectrum (MALDI-TOF) of partially chlorinated closo-dodecaborate Na₂B₁₂HₓCl₁₂₋ₓ.

FIG. 18: Chromatogram (HPLC-ELSD) of reaction mixture after 24 hours when CD6 is treated with CGTase in presence of partially chlorinated closo-dodecaborate template Na₂B₁₂HₓCl₁₂₋ₓ.

### DETAILED DESCRIPTION

### Definitions

By cyclodextrin glucanotransferase is meant an enzyme capable of catalysing the formation of cyclodextrins from starch, glucose, and similar substrates. Synonyms for and specific variants of cyclodextrin glucanotransferases include: 1,4-alpha-D-glucopyranosyl transferase; Akrilex C cyclodextrin glycosyltransferase; alpha-1,4-glucan 4-glycosyltransferase, cyclizing; alpha-cgt; alpha-CGTase; alpha-cyclodextrin glucanotransferase; alpha-cyclodextrin glucosyltransferase; alpha-cyclodextrin glycosyltransferase; Bacillus macerans amylase; beta-CGTase; beta-cyclodextrin glucanotransferase; beta-cyclodextrin glucosyltransferase; beta-cyclodextrin glycosyltransferase; beta-cyclodextrinase; BMA; C-CGTase; CD glucanotransferase; CGT; CGT13; CGTase; cgtS; CGTse ET1; CGT_TK; cyclodextrin beta-glucanotransferase; cyclodextrin glucanotransferase; cyclodextrin glucosyltransferase; cyclodextrin glycosyl transferase; cyclodextrin glycosyltransferase; cyclodextrin-glycosyltransferase; cyclodextrinase; cyclomaltodextrin glucanyltransferase; cyclomaltodextrin glucotransferase; cyclomaltodextrin glycanotransferase; cyclomaltodextrin glycosyltransferase; gamma-CGTase; gamma-cyclodextrin glycosyltransferase; konchizaimu; M-CGTase; neutral-cyclodextrin glycosyltransferase; PFCGT; and Toruzyme. The cyclodextrin glucanotransferase may be any enzyme defined in Enzyme Commission (EC) number 2.4.1.19 or 2.4.1.25.

The terms delta-cyclodextrin, delta-cyclomaltodextrin, and CD9 are used synonymously herein.

By delta-cyclodextrin derivative is meant a macrocyclic compound that consists substantially of 9 glucopyranose moieties, or a derivatives of glucopyranose. By this is meant that the ring does not consist of more than 9 or fewer than 9 pyranose moieties, but that the moieties do not strictly need to be glucopyranose, but can be derivatives thereof, for example by having one or more of the OH-groups substituted for other moieties. The person of skill in the art would readily recognise a delta-cyclodextrin derivative based on these considerations.

The following terms have the following meaning unless otherwise indicated. Any undefined terms have their art recognised meanings.

As used herein, the term “alkyl” by itself or as part of another substituent refers to a branched, or straight-chain monovalent hydrocarbon radical derived by the removal of one hydrogen atom from a single carbon atom of a parent alkane, alkene, alkyne, and the like. Where a specific degree of saturation is intended, the nomenclature “alkanyl”, “alkenyl”, and “alkynyl” is used. Typical alkyl groups include, but are not limited to, methyl, ethyl, propyls such as propan-1-yl or propan-2-yl; and butyls such as butan-1-yl, butan-2-yl, 2-methyl-propan-1-yl or 2-methyl-propan-2-yl, etc., and the like. In some embodiments, an alkyl group comprises from 1 to 20 carbon atoms. In other embodiments, an alkyl group comprises from 1 to 10 carbon atoms. In still other embodiments, an alkyl group comprises from 1 to 6 carbon atoms, such as from 1 to 4 carbon atoms.

“Alkanyl” by itself or as part of another substituent refers to a saturated branched, straight-chain or cyclic alkyl radical derived by the removal of one hydrogen atom from a single carbon atom of an alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methylpropan-1-yl (isobutyl), 2-methylpropan-2-yl (t-butyl), cyclobutan-1-yl, etc.; and the like.

“Alkylene” refers to a branched or unbranched saturated hydrocarbon chain, usually having from 1 to 40 carbon atoms, more usually 1 to 10 carbon atoms and even more usually 1 to 6 carbon atoms. This term is exemplified by groups such as methylene (—CH₂—), ethylene (—CH₂CH₂—), the propylene isomers (e.g., —CH₂CH₂CH₂- and —CH(CH₃)CH₂—) and the like.

“Alkenyl” by itself or as part of another substituent refers to an unsaturated branched, straight-chain or cyclic alkyl radical having at least one carbon-carbon double bond derived by the removal of one hydrogen atom from a single carbon atom of an alkene. The group may be in either the cis or trans conformation about the double bond(s). Typical alkenyl groups include, but are not limited to, ethenyl (vinyl); propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl” by itself or as part of another substituent refers to an unsaturated branched, straight-chain or cyclic alkyl radical having at least one carbon-carbon triple bond derived by the removal of one hydrogen atom from a single carbon atom of an alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Acyl” by itself or as part of another substituent refers to a radical —C(O)R^{a}, where R^{a} is hydrogen, alkyl, cycloalkyl, cycloheteroalkyl, aryl, arylalkyl, heteroalkyl, heteroaryl, heteroarylalkyl as defined herein and substituted versions thereof. Representative examples include, but are not limited to formyl, acetyl, cyclohexylcarbonyl, cyclohexylmethylcarbonyl, benzoyl, benzylcarbonyl, piperonyl, succinyl, and malonyl, and the like.

“Alkoxy” by itself or as part of another substituent refers to a radical —OR^{b} where R^{b} represents an alkyl or cycloalkyl group as defined herein. Representative examples include, but are not limited to, methoxy, ethoxy, propoxy, butoxy, cyclohexyloxy and the like.

“Alkoxycarbonyl” by itself or as part of another substituent refers to a radical —C(O)OR^{c} where R^{c} represents an alkyl or cycloalkyl group as defined herein. Representative examples include, but are not limited to, methoxycarbonyl, ethoxycarbonyl, propoxycarbonyl, butoxycarbonyl, cyclohexyloxycarbonyl and the like.

“Aryl” by itself or as part of another substituent refers to a monovalent aromatic hydrocarbon radical derived by the removal of one hydrogen atom from a single carbon atom of an aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, pentacene, pentalene, pentaphene, perylene, phenalene, phenanthrene, picene, pleiadene, pyrene, pyranthrene, rubicene, triphenylene, trinaphthalene and the like. In certain embodiments, an aryl group comprises from 6 to 20 carbon atoms. In certain embodiments, an aryl group comprises from 6 to 12 carbon atoms. Examples of an aryl group are phenyl and naphthyl. In one embodiment, “aryl” refers to “heteroaryl”.

“Arylalkyl” by itself or as part of another substituent refers to an alkyl radical in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-Naphthylethan-1-yl, 2-Naphthylethen-1-yl, naphthobenzyl, 2-Naphthophenylethan-1-yl and the like. In certain embodiments, an arylalkyl group is C₇-C₃₀ arylalkyl, e.g., the alkyl, alkenyl or alkynyl moiety of the arylalkyl group is C₁-C₁₀ and the aryl moiety is (C₆-C₂₀). In certain embodiments, an arylalkyl group is C₇-C₂₀ arylalkyl, e.g., the alkyl, alkenyl or alkynyl moiety of the arylalkyl group is (C₁-C₈) and the aryl moiety is (C₆-C₁₂).

“Arylaryl” by itself or as part of another substituent, refers to a monovalent hydrocarbon group derived by the removal of one hydrogen atom from a single carbon atom of a ring system in which two or more identical or non-identical aromatic ring systems are joined directly together by a single bond, where the number of such direct ring junctions is one less than the number of aromatic ring systems involved. Typical arylaryl groups include, but are not limited to, biphenyl, triphenyl, phenylnaphthyl, binaphthyl, biphenylnapthyl, and the like. When the number of carbon atoms in an arylaryl group are specified, the numbers refer to the carbon atoms comprising each aromatic ring. For example, C₅-C₁₄ arylaryl is an arylaryl group in which each aromatic ring comprises from 5 to 14 carbons, e.g., biphenyl, triphenyl, binaphthyl, phenylnapthyl, etc. In certain embodiments, each aromatic ring system of an arylaryl group is independently a C₅-C₁₄ aromatic. In certain embodiments, each aromatic ring system of an arylaryl group is independently a C₅-C₁₀ aromatic ring. In certain embodiments, each aromatic ring system is identical, e.g., biphenyl, triphenyl, binaphthyl, trinaphthyl, etc.

“Cycloalkyl” by itself or as part of another substituent refers to a saturated or unsaturated cyclic alkyl radical. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane and the like. In certain embodiments, the cycloalkyl group is C₃-C₁₀ cycloalkyl. In certain embodiments, the cycloalkyl group is C₃-C₇ cycloalkyl.

“Cycloheteroalkyl” or “heterocyclyl” by itself or as part of another substituent, refers to a saturated or unsaturated cyclic alkyl radical in which one or more carbon atoms (and any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Typical cycloheteroalkyl groups include, but are not limited to, groups derived from epoxides, azirines, thiiranes, imidazolidine, morpholine, piperazine, piperidine, pyrazolidine, pyrrolidine, quinuclidine and the like.

“Heteroalkyl, Heteroalkanyl, Heteroalkenyl and Heteroalkynyl” by themselves or as part of another substituent refer to alkyl, alkanyl, alkenyl and alkynyl groups, respectively, in which one or more of the carbon atoms (and any associated hydrogen atoms) are independently replaced with the same or different heteroatomic groups. Typical heteroatomic groups which can be included in these groups include, but are not limited to, —O—, —S—, —S—S—, —O—S—, —NR^{d}R^{e}—, =N—N═, —N═N—, —N═N—NR^{f}R^{g}—PR^{h}—, —P(O)₂—, —P(O)Rⁱ—, —OP(O)₂—, —S—O—, —S(O)—, —S(O)₂—, —SnR^{j}R^{k}— and the like, where R^{d}, R^{e}, R^{f}, R^{g}, R^{h}, Rⁱ, R^{j} and R^{k} are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl.

“Heteroaryl” by itself or as part of another substituent, refers to a monovalent heteroaromatic radical derived by the removal of one hydrogen atom from a single atom of a heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, benzodioxole and the like. In certain embodiments, the heteroaryl group is from 5-20 membered heteroaryl. In certain embodiments, the heteroaryl group is from 5-10 membered heteroaryl. In certain embodiments, heteroaryl groups are those derived from thiophene, pyrrole, benzothiophene, benzofuran, indole, pyridine, quinoline, imidazole, oxazole and pyrazine.

“Heteroarylalkyl” by itself or as part of another substituent, refers to an acyclic alkyl radical in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl and/or heterorylalkynyl is used. In certain embodiments, the heteroarylalkyl group is a 6-30 membered heteroarylalkyl, e.g., the alkanyl, alkenyl or alkynyl moiety of the heteroarylalkyl is 1-10 membered and the heteroaryl moiety is a 5-20-membered heteroaryl. In certain embodiments, the heteroarylalkyl group is 6-20 membered heteroarylalkyl, e.g., the alkanyl, alkenyl or alkynyl moiety of the heteroarylalkyl is 1-8 membered and the heteroaryl moiety is a 5-12-membered heteroaryl.

“Substituted” refers to a group in which one or more hydrogen atoms are independently replaced with the same or different substituent(s). Typical substituents include, but are not limited to, alkylenedioxy (such as methylenedioxy), -M, —Rⁱ, —O⁻, ═O, —OR^{l}, —SR^{l}, —S⁻, ═S, —NR^{l}R^{m}, =NR^{l}, —CF₃, —CN, —OCN, —SCN, —NO, —NO₂, ═N₂, —N₃, —S(O)₂O⁻, —S(O)₂OH, —S(O)₂R^{l}, —OS(O)₂O⁻, —OS(O)₂R^{l}, —P(O)(O⁻)₂, —P(O)(OR⁻)(O⁻), —OP(O)(OR^{l})(OR^{m}), —C(O)R^{l}, —C(S)R^{l}, —C(O)OR^{l}, —C(O)NR^{l}R^{m}, —C(O)O—, —C(S)OR^{l}, —NRⁿC(O)NR^{l}R^{m}, —NRⁿC(S)NR^{l}R^{m}, —NRⁿC(NR^{o})NR^{l}R^{m} and —C(NRⁿ)NR^{l}R^{m} where M is halogen; R^{l}, R^{m}, Rⁿ and R^{o} are independently hydrogen, alkyl, substituted alkyl, alkoxy, substituted alkoxy, cycloalkyl, substituted cycloalkyl, cycloheteroalkyl, substituted cycloheteroalkyl, aryl, substituted aryl, heteroaryl or substituted heteroaryl, or optionally R^{l} and R^{m} together with the nitrogen atom to which they are bonded form a cycloheteroalkyl or substituted cycloheteroalkyl ring. In certain embodiments, substituents include -M, —R^{l}, ═O, —OR^{l}, —SR^{l}, —S—, ═S, —NR^{l}R^{m}, ═NR^{l}, —CF₃, —CN, —OCN, —SCN, —NO, —NO₂, ═N₂, —N₃, —S(O)₂R^{l}, —OS(O)₂O—, —OS(O)₂R^{l}, —P(O)(O⁻)₂, —P(O)(OR^{l})(O⁻), —OP(O)(OR^{l})(OR^{m}), —C(O)R^{l}, —C(S)R^{l}, —C(O)OR⁻, —C(O)NR^{l}R^{m}, —C(O)O⁻, —NRⁿC(O)NR^{l}R^{m}. In certain embodiments, substituents include -M, —R^{l}, ═O, —OR^{l}, —SR^{l}, —NR^{l}R^{m}, —CF₃, —CN, —NO₂, —S(O)₂R^{l}, —P(O)(OR^{l})(O⁻), —OP(O)(OR^{l})(OR^{m}), —C(O)R^{l}, —C(O)OR^{l}, —C(O)NR^{l}R^{m}, —C(O)O⁻. In certain embodiments, substituents include -M, —R^{l}, ═O, —OR^{l}, —SR^{l}, —NR^{l}R^{m}, —CF₃, —CN, —NO₂, —S(O)₂R^{l}, —OP(O)(OR^{l})(OR^{m}), —C(O)R^{l}, —C(O)OR^{l}, —C(O)O⁻, where R^{l}, R^{m} and Rⁿ are as defined above. For example, a substituted group may bear a methylenedioxy substituent or one, two, or three substituents selected from a halogen atom, a C₁-C₄ alkyl group and a C₁-C₄ alkoxy group.

### Delta-Cyclodextrin and Derivatives Thereof

Allowing the enzyme cyclodextrin glycosyltransferase to act on a substrate containing a glucopyranose polysaccharide produces almost exclusively small-ring cyclodextrins, namely alpha-cyclodextrin, beta-cyclodextrin, and gamma-cyclodextrin, as kinetically trapped products. Very little large-ring cyclodextrin is formed. Isolating the large-ring cyclodextrin product formed requires extensive chromatography and provides for very low yields. The methods disclosed herein are capable of producing delta-cyclodextrin as the major cyclodextrin product in the reaction mixture, with little-to-no formation of other cyclodextrin product. This, in turn, allows for the isolation of delta-cyclodextrin in high yields.

Different cyclodextrins differing in size are difficult to separate, because they possess similar solubilities and hydrophilicities, making separation on column or by precipitation challenging if not impossible. Disclosed herein are methods for producing delta-cyclodextrin and derivatives thereof. The methods provide crude reaction mixtures containing delta-cyclodextrin or a derivative thereof as the major cyclodextrin product, such as wherein the delta-cyclodextrin or derivative thereof is the only cyclodextrin product. Accordingly, the methods disclosed herein allow for purification of the reaction mixtures using conventional precipitation/recrystallisation.

Thus, one embodiment of the present disclosure provides for a method for producing delta-cyclodextrin or a delta-cyclodextrin derivative, said method comprising mixing:
a. a glucose-based compound,
b. an enzyme such as cyclodextrin glucanotransferase, and
c. an ion of formula (B₁₂ClₙH₁₂₋ₙ)²⁻, wherein n is 1 to 12,
d. a solvent
to form a mixture, and
allowing the mixture to incubate,
thereby obtaining the delta-cyclodextrin or a delta-cyclodextrin derivative.

The methods disclosed herein have been shown useful in providing both delta-cyclodextrin, as well as derivatives thereof. Thus, in one embodiment, the delta-cyclodextrin or the delta-cyclodextrin derivative has the structure of formula I:

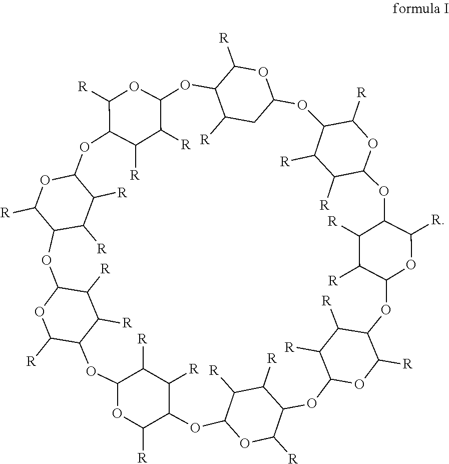

In one embodiment, the delta-cyclodextrin or the delta-cyclodextrin derivative has the structure of formula I, or a salt thereof,
wherein each R is independently selected from the group consisting of CH₂OR^{a}, CH₃, CH₂F, CH₂Cl, CH₂Br, CH₂I, CH₂N₃, CH₂CN, CH₂R^{a}, CH₂N(R^{a})₂, CH₂SR^{a}, CH₂Si(R^{a})₃, CH₂CHO, CH₂COOH, CH₂COOR^{a}, CH₂CON(R^{a})₂, CH₂CHNR^{a}, OR^{a}, H, F, Cl, Br, I, N₃, CN, R^{a}, N(R^{a})₂, SR^{a}, Si(R^{a})₃, CHO, COOH, COOR^{a}, CON(R^{a})₂, and CHNR^{a}, and wherein each R^{a} is independently selected from the group consisting of H, alkyl, substituted alkyl, alkenyl, substituted alkenyl, alkynyl, substituted alkynyl, acyl, substituted acyl, aryl, substituted aryl, benzyl, and substituted benzyl.

In one embodiment, the delta-cyclodextrin or the delta-cyclodextrin derivative has the structure of formula Ia:

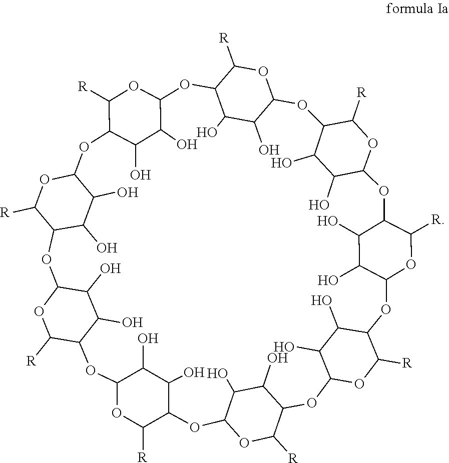

In one embodiment, the delta-cyclodextrin or the delta-cyclodextrin derivative has the structure of formula Ib:

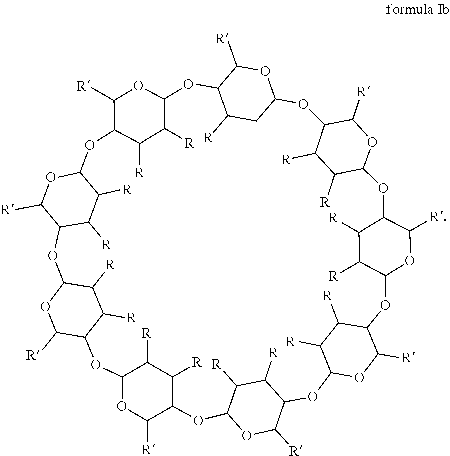

In one embodiment, the delta-cyclodextrin or the delta-cyclodextrin derivative has the structure of formula Ib,
wherein each R′ is independently selected from the group consisting of CH₂OR^{a}, CH₃, CH₂F, CH₂Cl, CH₂Br, CH₂I, CH₂N₃, CH₂CN, CH₂R^{a}, CH₂N(R^{a})₂, CH₂SR^{a}, CH₂Si(R^{a})₃, CH₂CHO, CH₂COOH, CH₂COOR^{a}, CH₂CON(R^{a})₂, CH₂CHNR^{a}, OR^{a}, H, F, Cl, Br, I, N₃, CN, R^{a}, N(R^{a})₂, SR^{a}, Si(R^{a})₃, CHO, COOH, COOR^{a}, CON(R^{a})₂, and CHNR^{a},
wherein each R is independently selected from the group consisting of OR^{a}, H, F, Cl, Br, I, N₃, CN, R^{a}, N(R^{a})₂, SR^{a}, Si(R^{a})₃, CHO, COOH, COOR^{a}, CON(R^{a})₂, and CHNR^{a}, and
wherein each R^{a} is independently selected from the group consisting of H, alkyl, substituted alkyl, alkenyl, substituted alkenyl, alkynyl, substituted alkynyl, acyl, substituted acyl, aryl, substituted aryl, benzyl, and substituted benzyl.

In one embodiment, the delta-cyclodextrin or the delta-cyclodextrin derivative has the structure of formula Ic:

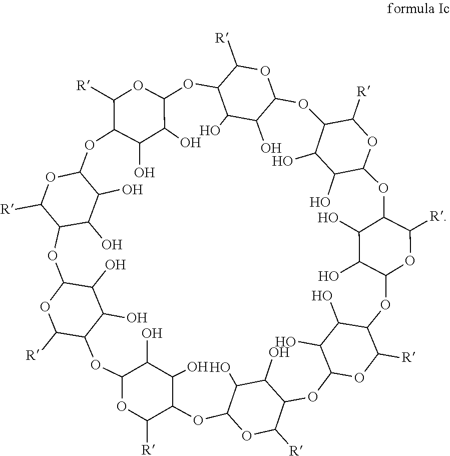

In one embodiment, R is independently selected from the group consisting of CH₂OR^{a}, CH₃, CH₂F, CH₂Cl, CH₂Br, CH₂I, CH₂N₃, CH₂CN, CH₂R^{a}, CH₂N(R^{a})₂, CH₂SR^{a}, CH₂Si(R^{a})₃, CH₂CHO, CH₂COOH, CH₂COOR^{a}, CH₂CON(R^{a})₂, CH₂CHNR^{a}, OR^{a}, H, F, Cl, Br, I, N₃, CN, R^{a}, N(R^{a})₂, SR^{a}, Si(R^{a})₃, CHO, COOH, COOR^{a}, CON(R^{a})₂, and CHNR^{a}, and
wherein each R^{a} is independently selected from the group consisting of H, alkyl, substituted alkyl, alkenyl, substituted alkenyl, alkynyl, substituted alkynyl, acyl, substituted acyl, aryl, substituted aryl, benzyl, and substituted benzyl The glucopyranose moieties making up the delta-cyclodextrin or derivative thereof may have differing substitution patterns, i.e., one or more of the glucopyranose moieties of the delta-cyclodextrin or derivatives thereof may differ. Thus, in one embodiment of the present disclosure, the delta-cyclodextrin or the delta-cyclodextrin derivative has the structure of formula I or formula Ib, and at least 1, 2, 3, 4, 5, 6, 7, 8, 9, 10, 11, 12, 13, 14, 15, 16, 17, 18, 19, 20, 21, 22, 23, 24, 25, 26, or 27 of the moieties R and/or R′ is/are selected from the group consisting of OR^{a}, H, F, Cl, Br, I, N₃, CN, R^{a}, N(R^{a})₂, SR^{a}, and Si(R^{a})₃.

In one embodiment of the present disclosure, the delta-cyclodextrin or the delta-cyclodextrin derivative has the structure of formula I or formula Ib, and at least 1, 2, 3, 4,5, 6, 7, 8, 9, 10, 11, 12, 13, 14, 15, 16, 17, 18, 19,20,21,22,23,24,25,26, or 27 of the moieties R and/or R′ is/are selected from the group consisting of OR^{a}, H, F, Cl, Br, I, N₃, CN, R^{a}, N(R^{a})₂, SR^{a}, and Si(R^{a})₃, and the remaining moieties are selected from the group consisting of CH₂OR^{a}, CH₃, CH₂F, CH₂Cl, CH₂Br, CH₂I, CH₂N₃, CH₂CN, CH₂R^{a}, CH₂N(R^{a})₂, CH₂SR^{a}, CH₂Si(R^{a})₃, CH₂CHO, CH₂COOH, CH₂COOR^{a}, CH₂CON(R^{a})₂, CH₂CHNR^{a}, OR^{a}, H, F, Cl, Br, I, N₃, CN, R^{a}, N(R^{a})₂, SR^{a}, Si(R^{a})₃, CHO, COOH, COOR^{a}, CON(R^{a})₂, and CHNR^{a}.

In one embodiment of the present disclosure, the delta-cyclodextrin derivative has the structure of formula Ia, and at least 1, 2, 3, 4, 5, 6, 7, 8, or 9 of the moiety R is/are selected from the group consisting of CH₂OR^{a}, CH₃, CH₂F, CH₂Cl, CH₂Br, CH₂I, CH₂N₃, CH₂CN, CH₂R^{a}, CH₂N(R^{a})₂, CH₂SR^{a}, CH₂Si(R^{a})₃, CH₂CHO, CH₂COOH, CH₂COOR^{a}, CH₂CON(R^{a})₂, CH₂CHNR^{a}, OR^{a}, H, F, Cl, Br, I, N₃, CN, R^{a}, N(R^{a})₂, SR^{a}, Si(R^{a})₃, CHO, COOH, COOR^{a}, CON(R^{a})₂, and CHNR^{a}.

In one embodiment of the present disclosure, the delta-cyclodextrin or the delta-cyclodextrin derivative has the structure of formula Ic, and at least 1, 2, 3, 4, 5, 6, 7, 8, or 9 of the moiety R′ is/are selected from the group consisting of CH₂OR^{a}, CH₃, CH₂F, CH₂Cl, CH₂Br, CH₂I, CH₂N₃, CH₂CN, CH₂R^{a}, CH₂N(R^{a})₂, CH₂SR^{a}, CH₂Si(R^{a})₃, CH₂CHO, CH₂COOH, CH₂COOR^{a}, CH₂CON(R^{a})₂, CH₂CHNR^{a}, OR^{a}, H, F, Cl, Br, I, N₃, CN, R^{a}, N(R^{a})₂, SR^{a}, Si(R^{a})₃, CHO, COOH, COOR^{a}, CON(R^{a})₂, and CHNR^{a}.

In one embodiment, the delta-cyclodextrin or the delta-cyclodextrin derivative has the structure of formula Ib, and at least 1, 2, 3, 4, 5, 6, 7, 8, 9, 10, 11, 12, 13, 14, 15, 16, 17, or 18 of the moiety R is/are selected from the group consisting of OR^{a}, H, F, Cl, Br, I, N₃, CN, R^{a}, N(R^{a})₂, SR^{a}, and Si(R^{a})₃.

In one embodiment, the delta-cyclodextrin or the delta-cyclodextrin derivative has the structure of formula Ia and at least 1, 2, 3, 4, 5, 6, 7, 8, or 9 of the moiety R is/are CH₂OH.

In one embodiment of the disclosure, the delta-cyclodextrin or the delta-cyclodextrin derivative has the structure of formula Ib and at least 1, 2, 3, 4, 5, 6, 7, 8, 9, 10, 11, 12, 13, 14, 15, 16, 17, or 18 of the moiety R is/are OH.

In one embodiment, the delta-cyclodextrin or the delta-cyclodextrin derivative has the structure of formula Ib and at least 1, 2, 3, 4, 5, 6, 7, 8, or 9 of the moiety R′ is/are CH₂OH.

In one embodiment of the present disclosure, the delta-cyclodextrin or the delta-cyclodextrin derivative has the structure of formula Ic and at least 1, 2, 3, 4, 5, 6, 7, 8, or 9 of the moiety R′ is/are CH₂OH.

In one embodiment, the delta-cyclodextrin or the delta-cyclodextrin derivative has the structure of formula Ib and at least 1, 2, 3, 4, 5, 6, 7, 8, or 9 of the moiety R′ is/are selected from the group consisting of CH₂OR^{a}, CH₃, CH₂F, CH₂Cl, CH₂Br, CH₂I, CH₂N₃, CH₂CN, CH₂R^{a}, CH₂N(R^{a})₂, CH₂SR^{a}, CH₂Si(R^{a})₃, CH₂CHO, CH₂COOH, CH₂COOR^{a}, CH₂CON(R^{a})₂, CH₂CHNR^{a}, OR^{a}, H, F, Cl, Br, I, N₃, CN, R^{a}, N(R^{a})₂, SR^{a}, Si(R^{a})₃, CHO, COOH, COOR^{a}, CON(R^{a})₂, and CHNR^{a}.

In one embodiment, the delta-cyclodextrin or the delta-cyclodextrin derivative has the structure of formula Ic and at least 1, 2, 3, 4, 5, 6, 7, 8, or 9 of the moiety R′ is/are selected from the group consisting of CH₂OR^{a}, CH₃, CH₂F, CH₂Cl, CH₂Br, CH₂I, CH₂N₃, CH₂CN, CH₂R^{a}, CH₂N(R^{a})₂, CH₂SR^{a}, CH₂Si(R^{a})₃, CH₂CHO, CH₂COOH, CH₂COOR^{a}, CH₂CON(R^{a})₂, CH₂CHNR^{a}, OR^{a}, H, F, Cl, Br, I, N₃, CN, R^{a}, N(R^{a})₂, SR^{a}, Si(R^{a})₃, CHO, COOH, COOR^{a}, CON(R^{a})₂, and CHNR^{a}.

In one embodiment of the present disclosure, the purity of the thereby obtained delta-cyclodextrin or derivative thereof is at least 50%, such as 55%, such as 60%, such as 65%, such as 70%, such as 75%, such as 80%, such as 85%, such as 90%, such as 91%, such as 92%, such as 93%, such as 94%, such at 95%, such as 96%, such as 97%, such as 98%, such as 99%. In one embodiment of the present disclosure, the isolated delta-cyclodextrin or derivative thereof comprises an ion of (B₁₂ClₙH₁₂₋ₙ)²⁻.

In one embodiment, the disclosed method provides an isolated yield of the delta-cyclodextrin or derivative thereof of at least 1%, such as 2%, such as 3%, such as 4%, such as 5%, such as 6%, such as 7%, such as 8%, such as 9%, such as 10%, such as 15%, such as 20%, such as 25%, such as 30%, such as 35%, such at 40%, such as 45%, such as 50%.

### Glucose-Based Compounds

As shown in the examples herein, the method disclosed herein is capable of producing delta-cyclodextrin or derivatives thereof starting from other cyclodextrins and/or linear glucopyranose species. Thus, in one embodiment the glucose-based compound is linear or cyclic. By glucose-based compound is preferably meant a compound comprising one or more glucopyranose moieties. By glucose-based compound is preferably meant a compound comprising glucopyranose moieties linked with 1,4-bonds.

In one embodiment of the present disclosure, the glucose-based compound has the structure of formula II:

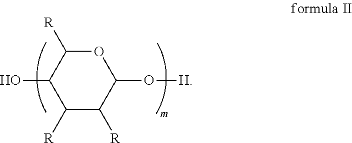

In one embodiment of the present disclosure, the glucose-based compound has the structure of formula II,
wherein each R is independently selected from the group consisting of CH₂OR^{a}, CH₃, CH₂F, CH₂Cl, CH₂Br, CH₂I, CH₂N₃, CH₂CN, CH₂R^{a}, CH₂N(R^{a})₂, CH₂SR^{a}, CH₂Si(R^{a})₃, CH₂CHO, CH₂COOH, CH₂COOR^{a}, CH₂CON(R^{a})₂, CH₂CHNR^{a}, OR^{a}, H, F, Cl, Br, I, N₃, CN, R^{a}, N(R^{a})₂, SR^{a}, Si(R^{a})₃, CHO, COOH, COOR^{a}, CON(R^{a})₂, and CHNR^{a},
wherein each R^{a} is independently selected from the group consisting of H, alkyl, substituted alkyl, alkenyl, substituted alkenyl, alkynyl, substituted alkynyl, acyl, substituted acyl, aryl, substituted aryl, benzyl, and substituted benzyl, and wherein m is an integer 2 or more.

Cyclodextrin glucanotransferases (CGTases) can act on substrates comprising two or more glucopyranose subunits, such as substrates comprising two or more glucopyranose subunits linked with a 1,4-glycosylic bond. Thus, in one embodiment, m is an integer 2 or more.

In one embodiment, the glucose-based compound has the structure of formula Ila:

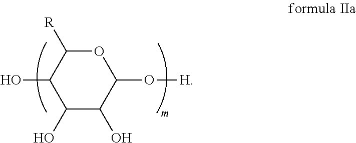

In one embodiment, the glucose-based compound has the structure of formula |Ib:

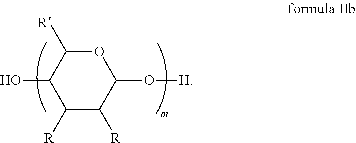

In one embodiment, the glucose-based compound has the structure of formula |Ib,
wherein each R′ is independently selected from the group consisting of CH₂OR^{a}, CH₃, CH₂F, CH₂Cl, CH₂Br, CH₂I, CH₂N₃, CH₂CN, CH₂R^{a}, CH₂N(R^{a})₂, CH₂SR^{a}, CH₂Si(R^{a})₃, CH₂CHO, CH₂COOH, CH₂COOR^{a}, CH₂CON(R^{a})₂, CH₂CHNR^{a}, OR^{a}, H, F, Cl, Br, I, N₃, CN, R^{a}, N(R^{a})₂, SR^{a}, Si(R^{a})₃, CHO, COOH, COOR^{a}, CON(R^{a})₂, and CHNR^{a},
wherein each R is independently selected from the group consisting of OR^{a}, H, F, Cl, Br, I, N₃, CN, R^{a}, N(R^{a})₂, SR^{a}, and Si(R^{a})₃,
wherein each R^{a} is independently selected from the group consisting of H, alkyl, substituted alkyl, alkenyl, substituted alkenyl, alkynyl, substituted alkynyl, acyl, substituted acyl, aryl, substituted aryl, benzyl, and substituted benzyl, and wherein m is an integer 2 or more.

In one embodiment, the glucose-based compound has the structure of formula Ic:

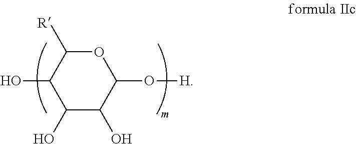

As shown in the Examples herein, the disclosed methods are capable of converting other cyclodextrins to delta-cyclodextrins or derivatives thereof. Such other cyclodextrins may be alpha-cyclodextrin, beta-cyclodextrin, gamma-cyclodextrin, or dextrins having a ring size of 10 or more. Thus, in one embodiment, the glucose-based compound has the structure of formula III:

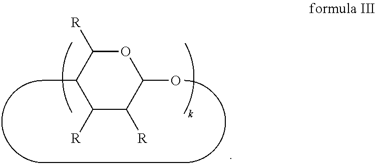

In one embodiment, the glucose-based compound has the structure of formula III,
wherein each R is independently selected from the group consisting of CH₂OR^{a}, CH₃, CH₂F, CH₂Cl, CH₂Br, CH₂I, CH₂N₃, CH₂CN, CH₂R^{a}, CH₂N(R^{a})₂, CH₂SR^{a}, CH₂Si(R^{a})₃, CH₂CHO, CH₂COOH, CH₂COOR^{a}, CH₂CON(R^{a})₂, CH₂CHNR^{a}, OR^{a}, H, F, Cl, Br, I, N₃, CN, R^{a}, N(R^{a})₂, SR^{a}, Si(R^{a})₃, CHO, COOH, COOR^{a}, CON(R^{a})₂, and CHNR^{a},
wherein each R^{a} is independently selected from the group consisting of H, alkyl, substituted alkyl, alkenyl, substituted alkenyl, alkynyl, substituted alkynyl, acyl, substituted acyl, aryl, substituted aryl, benzyl, and substituted benzyl, and wherein k is an integer 6, 7, 8, 10, or more than 10.

The structure depicted with formula III and the like is meant to illustrate a macrocycle of 1,4-linked pyranose moieties.

In one embodiment of the disclosure, k is an integer 6, 7, or 8. In one embodiment, k is an integer 10 or more than 10.

In one embodiment, the glucose-based compound has the structure of formula IIIa:

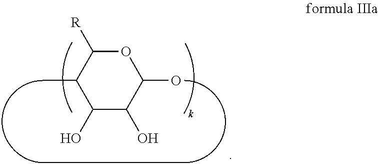

In one embodiment, the glucose-based compound has the structure of formula IIIb:

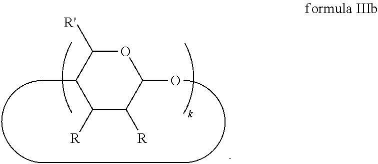

In one embodiment, the glucose-based compound has the structure of formula IIIb.
wherein each R′ is independently selected from the group consisting of CH₂OR^{a}, CH₃, CH₂F, CH₂Cl, CH₂Br, CH₂I, CH₂N₃, CH₂CN, CH₂R^{a}, CH₂N(R^{a})₂, CH₂SR^{a}, CH₂Si(R^{a})₃, CH₂CHO, CH₂COOH, CH₂COOR^{a}, CH₂CON(R^{a})₂, CH₂CHNR^{a}, OR^{a}, H, F, Cl, Br, I, N₃, CN, R^{a}, N(R^{a})₂, SR^{a}, Si(R^{a})₃, CHO, COOH, COOR^{a}, CON(R^{a})₂, and CHNR^{a},
wherein each R is independently selected from the group consisting of OR^{a}, H, F, Cl, Br, I, N₃, CN, R^{a}, N(R^{a})₂, SR^{a}, and Si(R^{a})₃,
wherein each R^{a} is independently selected from the group consisting of H, alkyl, substituted alkyl, alkenyl, substituted alkenyl, alkynyl, substituted alkynyl, acyl, substituted acyl, aryl, substituted aryl, benzyl, and substituted benzyl, and
wherein k is an integer 6, 7, 8, 10, or more than 10.

In one embodiment, the glucose-based compound has the structure of formula IIIc:

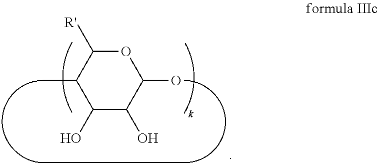

In one embodiment of the present disclosure, each R is independently selected from the group consisting of CH₂OR^{a}, CH₃, CH₂F, CH₂Cl, CH₂Br, CH₂I, CH₂N₃, CH₂CN, CH₂R^{a}, CH₂N(R^{a})₂, CH₂SR^{a}, CH₂Si(R^{a})₃, CH₂CHO, CH₂COOH, CH₂COOR^{a}, CH₂CON(R^{a})₂, CH₂CHNR^{a}, OR^{a}, H, F, Cl, Br, I, N₃, CN, R^{a}, N(R^{a})₂, SR^{a}, Si(R^{a})₃, CHO, COOH, COOR^{a}, CON(R^{a})₂, and CHNR^{a}, and
wherein each R^{a} is independently selected from the group consisting of H, alkyl, substituted alkyl, alkenyl, substituted alkenyl, alkynyl, substituted alkynyl, acyl, substituted acyl, aryl, substituted aryl, benzyl, and substituted benzyl.

The mixture described herein may comprise more than one glucose-based compound. Thus, in one embodiment of the present disclosure, the mixture further comprises a second glucose-based compound.

In one embodiment, the mixture further comprises a second glucose-based compound having the structure of formula IV:

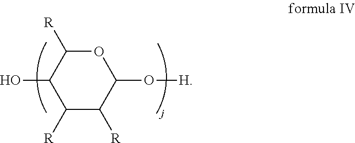

In one embodiment, the mixture further comprises a second glucose-based compound having the structure for formula IV,
wherein each R is independently selected from the group consisting of CH₂OR^{a}, CH₃, CH₂F, CH₂Cl, CH₂Br, CH₂I, CH₂N₃, CH₂CN, CH₂R^{a}, CH₂N(R^{a})₂, CH₂SR^{a}, CH₂Si(R^{a})₃, CH₂CHO, CH₂COOH, CH₂COOR^{a}, CH₂CON(R^{a})₂, CH₂CHNR^{a}, OR^{a}, H, F, C, Br, I, N₃, CN, R^{a}, N(R^{a})₂, SR^{a}, Si(R^{a})₃, CHO, COOH, COOR^{a}, CON(R^{a})₂, and CHNR^{a},
wherein each R^{a} is independently selected from the group consisting of H, alkyl, substituted alkyl, alkenyl, substituted alkenyl, alkynyl, substituted alkynyl, acyl, substituted acyl, aryl, substituted aryl, benzyl, and substituted benzyl, and wherein j is an integer 1 or more.

In one embodiment of the present disclosure, the second glucose-based compound has the structure of formula IVa:

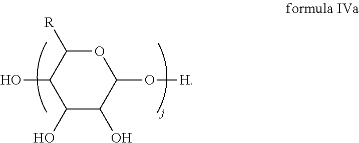

In one embodiment of the disclosure, the second glucose-based compound has the structure of formula IVb:

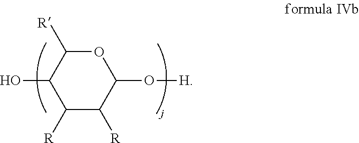

In one embodiment of the disclosure, the second glucose-based compound has the structure of formula IVb,
wherein each R′ is independently selected from the group consisting of CH₂OR^{a}, CH₃, CH₂F, CH₂Cl, CH₂Br, CH₂I, CH₂N₃, CH₂CN, CH₂R^{a}, CH₂N(R^{a})₂, CH₂SR^{a}, CH₂Si(R^{a})₃, CH₂CHO, CH₂COOH, CH₂COOR^{a}, CH₂CON(R^{a})₂, CH₂CHNR^{a}, OR^{a}, H, F, Cl, Br, I, N₃, CN, R^{a}, N(R^{a})₂, SR^{a}, Si(R^{a})₃, CHO, COOH, COOR^{a}, CON(R^{a})₂, and CHNR^{a},
wherein each R is independently selected from the group consisting of OR^{a}, H, F, Cl, Br, I, N₃, CN, R^{a}, N(R^{a})₂, SR^{a}, and Si(R^{a})₃,
wherein each R^{a} is independently selected from the group consisting of H, alkyl, substituted alkyl, alkenyl, substituted alkenyl, alkynyl, substituted alkynyl, acyl, substituted acyl, aryl, substituted aryl, benzyl, and substituted benzyl, and
wherein j is an integer 1 or more.

In one embodiment, the second glucose-based compound has the structure of formula IVc:

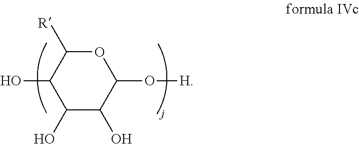

In one embodiment of the present disclosure, each R′ is independently selected from the group consisting of CH₂OR^{a}, CH₃, CH₂F, CH₂Cl, CH₂Br, CH₂I, CH₂N₃, CH₂CN, CH₂R^{a}, CH₂N(R^{a})₂, CH₂SR^{a}, CH₂Si(R^{a})₃, CH₂CHO, CH₂COOH, CH₂COOR^{a}, CH₂CON(R^{a})₂, CH₂CHNR^{a}, OR^{a}, H, F, Cl, Br, I, N₃, CN, R^{a}, N(R^{a})₂, SR^{a}, Si(R^{a})₃, CHO, COOH, COOR^{a}, CON(R^{a})₂, and CHNR^{a}. In one embodiment of the present disclosure, each R is independently selected from the group consisting of OR^{a}, H, F, Cl, Br, I, N₃, CN, R^{a}, N(R^{a})₂, SR^{a}, and Si(R^{a})₃. In one embodiment of the present disclosure, each R^{a} is independently selected from the group consisting of H, alkyl, substituted alkyl, alkenyl, substituted alkenyl, alkynyl, substituted alkynyl, acyl, substituted acyl, aryl, substituted aryl, benzyl, and substituted benzyl. In one embodiment of the present disclosure, j is an integer 1 or more.

In one embodiment, the glucose-based compound is selected from the group consisting of: glucose, alpha-cyclodextrin, beta-cyclodextrin, gamma-cyclodextrin, a second cyclodextrin, a second large-ring cyclodextrin, amylose, synthetic amylose, glycogen, maltodextrin, dextrin, cycloamylose, amylopectin, starch, modified starch such as soluble starch, limit-dextrin and other alpha-glucans such as pullulan or any mixture of all of the above. In the context of the substrate, by a large-ring cyclodextrin is meant a cyclodextrin consisting of a 10 or more glucopyranose moieties.

In one embodiment, the glucose-based compound is glucose, a glucose derivative, a modified glucose, a glucose-based polysaccharide, a glucose-based polysaccharide derivative, a modified glucose-based polysaccharide, a second cyclodextrin, or a second cyclodextrin derivative.

In one embodiment, the second cyclodextrin is alpha-cyclodextrin, beta-cyclodextrin, or gamma-cyclodextrin. In one embodiment, the second large-ring cyclodextrin is a cyclodextrin having a size of 10 glucose moieties or more, or a derivative of a cyclodextrin having a size of 10 glucose moieties or more.

In one embodiment, the second cyclodextrin derivative is an alpha-cyclodextrin derivative, a beta-cyclodextrin derivative, or a gamma-cyclodextrin derivative. In one embodiment, the second cyclodextrin derivative is a modified cyclodextrin, such as a modified alpha-cyclodextrin, a modified beta-cyclodextrin, or a modified gamma-cyclodextrin.

As shown in the examples, the presently disclosed method is capable of producing delta-cyclodextrin derivatives by using modified glucose-based compounds. In one embodiment the modification is a substitution of one or more of the OH moieties present on the glucose-based compound by a moiety independently selected from the group consisting of OR^{a}, H, F, Cl, Br, I, N₃, CN, R^{a}, N(R^{a})₂, SR^{a}, Si(R^{a})₃, and/or wherein the modification is a substitution of one or more of the CH₂OH moieties present on the glucose-based compound by a moiety independently selected from the group consisting of CH₂OR^{a}, CH₃, CH₂F, CH₂Cl, CH₂Br, CH₂I, CH₂N₃, CH₂CN, CH₂R^{a}, CH₂N(R^{a})₂, CH₂SR^{a}, CH₂Si(R^{a})₃, CH₂CHO, CH₂COOH, CH₂COOR^{a}, CH₂CON(R^{a})₂, CH₂CHNR^{a}, OR^{a}, H, F, Cl, Br, I, N₃, CN, R^{a}, N(R^{a})₂, SR^{a}, Si(R^{a})₃, CHO, COOH, COOR^{a}, CON(R^{a})₂, and CHNR^{a},
wherein each R^{a} is independently selected from the group consisting of H, alkyl, substituted alkyl, alkenyl, substituted alkenyl, alkynyl, substituted alkynyl, acyl, substituted acyl, aryl, substituted aryl, benzyl, and substituted benzyl.

In a specific embodiment, the modified cyclodextrin derivative is a deoxy cyclodextrin, such as a monodeoxy cyclodextrin, such as a 6-deoxy cyclodextrin, such as 6-deoxy alpha-cyclodextrin, such as mono-6-deoxy alpha-cyclodextrin.

### Template

The method of the disclosure is a so-called templated synthesis, wherein a reactant does not necessarily form covalent bonds to the other reactants, but rather directs formation of product via non-covalent interactions to the other reaction partners. The present inventors have discovered that certain boron-halogen clusters are capable of templating the synthesis of delta-cyclodextrin. A template can effect formation of a specific product for example by stabilising the desired product, i.e., it induces formation of the product by making its formation more thermodynamically favourable. A template can also affect reaction kinetics, making formation of a certain product more favourable by increasing the rate at which it forms. In a specific embodiment, it is contemplated that the presently disclosed method is governed by a thermodynamic templating effect.

One embodiment of the present disclosure provides for an ion of formula (B₁₂ClₙH₁₂₋ₙ)²⁻. In one embodiment, n is 1 to 12. In one embodiment, n is 1, 2, 3, 4, 5, 6, 7, 8, 9, 10, 11, or 12. In one embodiment, n is 9, 10, 11, or 12. In one embodiment, the ion of formula [B₁₂ClₙH₁₂₋ₙ]²⁻ is selected from the group consisting of (B₁₂Cl₁₂)²⁻, (B₁₂Cl₁₁H)²⁻, (B₁₂Cl₁₀H₂)²⁻, (B₁₂Cl₉H₃)²⁻, (B₁₂Cl₈H₄)²⁻, (B₁₂Cl₇H₅)²⁻, (B₁₂Cl₆H₆)²⁻, (B₁₂Cl₅H₇)²⁻, (B₁₂Cl₄H₈)²⁻, (B₁₂Cl₃H₉)²⁻, (B₁₂Cl₂H₁₀)²⁻, and (B₁₂ClH₁₁)²⁻. In one embodiment, the ion of formula [B₁₂ClₙH₁₂₋ₙ]²⁻ is selected from the group consisting of (B₁₂Cl₁₂)²⁻, (B₁₂Cl₁₁H)²⁻, (B₁₂Cl₁₀H₂)²⁻, and (B₁₂Cl₉H₃)²⁻. In one embodiment, the ion of formula (B₁₂ClₙH₁₂₋ₙ)²⁻ is (B₁₂Cl₁₂)²⁻.

In one embodiment of the disclosure, the ion of formula (B₁₂ClₙH₁₂₋ₙ)²⁻ is from a salt of formula M₂(B₁₂ClₙH₁₂₋ₙ) or M(B₁₂ClₙH₁₂₋ₙ), wherein M is a monovalent or a divalent cation. In one embodiment, M is selected from the group consisting of alkali metal ions such as Li⁺, Na⁺, K⁺, Cs⁺, earth-alkali metal ions such as Mg²⁺ and Ca²⁺, NH₄⁺, (R^{p})₄N⁺, and (R^{P})₄P⁺,
wherein each RP is independently selected from the group consisting of H, alkyl, substituted alkyl, alkenyl, substituted alkenyl, alkynyl, substituted alkynyl, acyl, substituted acyl, aryl, substituted aryl, benzyl, and substituted benzyl.

In one embodiment, the salt of formula M₂(B₁₂ClₙH₁₂₋ₙ) is Na₂(B₁₂Cl₁₂).

### Enzyme

In one embodiment of the present disclosure, the enzyme such as the cyclodextrin glucanotransferase is a cyclodextrin glucanotransferase as defined by Enzyme Commission (EC) number 2.4.1.19. Other enzymes might be capable of catalysing the hydrolysis and/or condensation of 1,4-glycosidic bonds. Thus, in one embodiment, the enzyme, such as cyclodextrin glucanotransferase is an enzyme capable of catalysing the reactions catalysed by enzymes defined by Enzyme Commission (EC) number 2.4.1.19. In one embodiment, the enzyme such as the cyclodextrin glucanotransferase is selected from the group of enzymes defined by Enzyme Commission (EC) number 2.4.1.25. In one embodiment, the cyclodextrin glucanotransferase is selected from the group of enzymes defined by Enzyme Commission (EC) number 3.2.1.54. In one embodiment, the cyclodextrin glucanotransferase is CGTase, catalogue number 970390 from Amano Enzyme Europe Limited. In one embodiment, the enzyme is cyclodextrin glucanotransferase as disclosed herein.

In one embodiment, the cyclodextrin glucanotransferase is the cyclodextrin glucanotransferase of Alkalihalobacillus alcalophilus, of Alkalihalobacillus clausii, of Alkalihalobacillus lehensis, of Alkalihalobacillus pseudalcaliphilus, of Anaerobranca gottschalkii, of Bacillus autolyticus, of Bacillus cereus, of Bacillus licheniformis, of Bacillus ohbensis, of Bacillus sp., of Bacillus subtilis, of Brevibacillus brevis, of Brevibacterium sp., of Cytobacillus firmus, of E. coli, of Evensella clarkii, of Geobacillus stearothermophilus, of Haloferax mediterranei, of Klebsiella oxytoca, of Klebsiella pneumoniae, of Lederbergia lentus, of Lysinibacillus sphaericus, of Microbacterium terrae, of Micrococcus luteus, of Micrococcus sp., of Niallia circulans, of Nostoc sp., of Paenibacillus barengoltzii, of Paenibacillus compinasensis, of Paenibacillus graminis, of Paenibacillus illinoisensis, of Paenibacillus macerans, of Paenibacillus pabuli, of Paenibacillus sp., of Parageobacillus thermoglucosidasius, of Priestia flexa, of Priestia megaterium, of Pyrococcus furiosus, of Saccharomyces cerevisiae, of Salimicrobium halophilum, of Salipaludibacillus agaradhaerens, of Streptococcus pyogenes, of Sus scrofa, of Thermoactinomyces vulgaris, of Thermoanaerobacter sp., of Thermoanaerobacterium thermosulfurigenes, of Thermococcus kodakarensis, of Thermococcus sp., of Trichoderma viride, of Carboxydocella sp., of Weizmannia coagulans, of Xanthomonas axonopodis, or of Xanthomonas campestris. In a specific embodiment, the cyclodextrin glucanotransferase is the cyclodextrin glucanotransferase of Paenibacillus macerans.

In one embodiment, the enzyme is a 4-alpha-glucanotransferase. In one embodiment, the 4-alpha-glucanotransferase is the 4-alpha-glucanotransferase of Acidothermus celluloluticus, of Aquifex aeolicus, of Arabidopsis thaliana, of Aspergillus niger, of Bacillus amyloloquefaciens, of Bacillus sp., of Bacillus subtilis, of Borreliella burgdorferi, of Bos taurus, of Chlamudia pneumoniae, of Chlamydia psittaci, of Chlamydomonas reinhardtii, of Clostridium butyricum, of Corynebacterium glutamicum, of Culex quinquefasciatus, of Daucus carota, of Dictyoglumus thermophilum, of E. coli, of Gallus gallus, of Haemophilus influenzae, of Homo sapiens, of Hordeum vulgare, of Ipomoea batatas, of Manihot esculenta, of Mycobacterium tuberculosis, of Oryctolagus cuniculus, of Oryza sativa, of Paenibacillus macerans, of Pisum sativum, of Pseudomonas stutzeri, of Pyrobaculum aerophilum, of Pyrococcus furiosus, of Pyrococcus sp., of Rattus norvegicus, of Saccharolobus sulfataricus, of Saccharomyces cerevisiae, of Solanum lycopersicum, of Solanum tuberosum, of Spinacia oleracea, of Squalus acanthias, of Streptococcus equinus, of Streptococcus mitis, of Streptococcus mutans, of Streptococcus pneumoniae, of Streptococcus sp., of Sus scrofa, of Synechocystis sp., of Thermococcus kodakarensis, of Thermococcus literalis, of Thermotoga maritima, of Thermotoga neapolitana, of Thermus aquaticus, of Thermus brockianus, of Thermus filiformis, of Thermus Scotoductus, of Thermus thermophilus, of Triticum aestivum, of Vicia faba, of Vigna radiate, or of Zea mays. In one embodiment, the disclosed method is an in vitro method.

In one embodiment, the enzyme is a cyclomaltodextrinase. In one embodiment, the cyclomaltodextrinase is the cyclomaltodextrinase of Alicyclobacillus acidocaldarius, of Anoxybacillus flavithermus, of Archaeoglobus fulgidus, of Bacillus licheniformis, of Bacillus sp., of Bacillus subtilis, of Bacillus thermoalkalophilus, of Bacteroides ovatus, of Escherichia coli, of Evansella clarkia, of Flavobacterium sp., of Geobacillus stearothermophilus, of Klebsiella oxytoca, of Laceyella sacchari, of Lactobacillus sp., of Lactococcus lactis, of Lysinibacillus sphaericus, of Massilia timonae, of Niallia circulans, of Nostoc punctiforme, of Paenibacillus macerans, of Paenibacillus sp., of Palaeococcus pacificus, of Parabacteroides distasonis, of Pyrococcus furiosus, of Thermoactinomyces vulgaris, of Thermoanaerobacter ethanolicus, of Thermoanaerobacter thermohydrosulfuricus, of Thermococcus kodakarensis, of Thermococcus sp., of Thermofilum pendens, of Thermoplasma volcanium, of Thermotoga maritima, of Thermotoga neapolitana, of Thermus, of Weizmannia coagulans, or of Xanthomonas campestris.

### Solvent

The present method is carried out in a solvent. In one embodiment, the solvent is a protic solvent. In one embodiment, the solvent is a polar solvent. In one embodiment, the solvent is a protic, polar solvent. In one embodiment, the solvent comprises two or more solvents. Solvent mixtures may be employed for the presently disclosed method. Thus, in one embodiment, the solvent comprises water and an organic solvent. The organic solvent may be polar and/or protic. In a specific embodiment, the solvent comprises or is dimethylformamide, dimethylsulfoxide, acetone, acetonitrile, isopropanol, ethanol, or methanol.

In one embodiment, the solvent comprises or is an aqueous solvent. In one embodiment, the aqueous solvent comprises or is water, such as deionised water, for example MilliQ water.

In one embodiment, the solvent comprises or is an aqueous buffer. In one embodiment, the aqueous buffer is a phosphate buffer or a sulphate buffer. In one embodiment, the concentration of the aqueous buffer is 1 to 500 mM, such as 1 to 250 mM, such as 5 to 150 mM, such as 10 to 100 mM, such as 20 to 100 mM, such as about 50 mM.

In one embodiment, the solvent has a pH of 3.5 to 11.0, such as 4.0 to 9.0, such as 4.5 to 9.0, such as 5.5 to 8.5, such as 5.5 to 8.5, such as 6.0 to 8.5, such as 6.5 to 8.5, such as 7.0 to 8.0, such as about 7.5. In one embodiment, the solvent has a pH within the working pH range of the enzyme, such as the cyclodextrin glucanotransferase. In one embodiment, the solvent has a pH of 4.0 to 4.5, such as 4.5 to 5.0, such as 5.0 to 5.5, such as 5.5 to 6.0, such as 6.0 to 6.5, such as 6.5 to 7.0, such as 7.0 to 7.5, such as 7.5 to 8.0, such as 8.0 to 8.5, such as 8.5 to 9.0. In one embodiment, the mixture has a pH of, or is pH-adjusted to 3.5 to 11.0, such as 4.0 to 9.0, such as 4.5 to 9.0, such as 5.5 to 8.5, such as 5.5 to 8.5, such as 6.0 to 8.5, such as 6.5 to 8.5, such as 7.0 to 8.0, such as about 7.5. In one embodiment, the mixture has a pH or is adjusted to a pH within the working pH range of the enzyme, such as the cyclodextrin glucanotransferase. In one embodiment, the mixture has a pH of or is pH adjusted to 4.0 to 4.5, such as 4.5 to 5.0, such as 5.0 to 5.5, such as 5.5 to 6.0, such as 6.0 to 6.5, such as 6.5 to 7.0, such as 7.0 to 7.5, such as 7.5 to 8.0, such as 8.0 to 8.5, such as 8.5 to 9.0.

### Incubation

Incubation is carried out under conditions and until sufficient product has been produced.

In one embodiment of the present disclosure, the incubation is carried out at 0 to 120° C., such as 0 to 100° C., such as 0 to 80° C., such as 0 to 70° C., such as 0 to 60° C., such as 5 to 45° C., 10 to 40° C., such as 15 to 40° C., such as 20 to 35° C. In one embodiment, wherein the incubation is carried out at 0 to 5° C., such as 5 to 10° C., such as 10 to 15° C., such as 15 to 20° C., such as 20 to 25° C., such as 25 to 30° C., such as 30 to 35° C., such as 35 to 40° C., such as 40 to 45° C.

In one embodiment, the incubation is carried out for 10 to 200 hours, such as 10 to 150 hours, such as 10 to 100 hours, such as 10 to 80 hours, such as 20 to 70 hours, such as 30 to 60 hours, such as 35 to 55 hours, such as about 40 to 50 hours. In one embodiment, the incubation is carried out for 10 to 15 hours, such as 15 to 20 hours, such as 20 to 25 hours, such as 25 to 30 hours, such as 30 to 35 hours, such as 35 to 40, such as 40 to 45 hours, such as 45 to 50 hours, such as 50 to 55 hours, such as 55 to 60 hours, such as 60 to 65 hours, such as 65 to 70 hours, such as 70 to 75 hours, such as 75 to 80 hours, such as 80 to 85 hours, such 85 to 90 hours, such as 90 to 95 hours, such as 95 to 100 hours. In a specific embodiment, the incubation is carried out for at least 24 hours, such as at least 36 hours, such as at least 42 hours, such as at least 48 hours. In a specific embodiment, the incubation is carried out for about 42 hours or about 48 hours.

The present inventors contemplate that the optimal incubation time for the disclosed method will vary, depending on incubation temperature and the concentration of the enzyme, the glucose-based compound, and the template. The person of skill in the art will know how to follow the progress of the reaction using methods well-known to them, and shown in the examples, in order to determine when to stop the incubation.

### Reagent Stoichiometry

As outlined herein, the ion of formula (B₁₂ClₙH₁₂₋ₙ)²⁻ acts as a template, and accordingly, does not necessarily need to be present in stoichiometric amounts compared to the delta-cyclodextrin or derivative thereof, nor to the product delta-cyclodextrin or derivative thereof.

In one embodiment of the present disclosure, the ratio of glucose-based compound to ion of formula (B₁₂ClₙH₁₂₋ₙ)²⁻ is between 99:1 and 20:80 (w/w), such as between 90:10 and 65:35.

In one embodiment of the present disclosure, the ratio of glucose-based compound to ion of formula (B₁₂ClₙH₁₂₋ₙ)²⁻ is 99:1 to 98:2 (w/w), such as 98:2 to 95:5, such as 95:5 to 90:10, such as 90:10 to 85:15, such as 85:15 to 80:20, such as 80:20 to 75:25, such as 75:25 to 70:30, such as 70:30 to 65:35, such as 65:35 to 60:40, such as 60:40 to 55:45, such as 55:45 to 50:50, such as 50:50 to 45:55, such as 45:55 to 40:60, such as 40:60 to 35:65, such as 35:65 to 30:70, such as 30:70 to 25:75, such as 25:75 to 20:80.

In one embodiment, the amount of ion of formula (B₁₂ClₙH₁₂₋ₙ)²⁻ is 2 to 50 mol % per monosaccharide moiety in the glucose-based compound, such as 2 to 45 mol %, such as 3 to 40 mol %, such as 4 to 35 mol %, such as 5 to 30 mol %, such as 6 to 25 mol %.

In one embodiment, the amount of ion of formula (B₁₂ClₙH₁₂₋ₙ)²⁻ is 2 to 3 mol %, such as 3 to 4 mol %, such as 4 to 5 mol %, such as 5 to 6 mol %, such as 6 to 7 mol %, such as 7 to 8 mol %, such as 8 to 9 mol %, such as 9 to 10 mol %, such as 10 to 11 mol %, such as 11 to 12 mol %, such as 12 to 13 mol %, such as 13 to 14 mol %, such as 14 to 15 mol %, such as 15 to 17 mol %, such as 17 to 20 mol %, such as 20 to 25 mol %, such as 25 to 30 mol %, such as 30 to 35 mol %, such as 35 to 40 mol %, such as 40 to 45 mol %, such as 45 to 50 mol %.

In one embodiment, the mixture comprises an amount of cyclodextrin glucanotransferase corresponding to 0.1 to 100% (v/v) cyclodextrin glucanotransferase stock solution, such as 0.1 to 60%, such as 0.1 to 50%, such as 0.5 to 40%, such as 0.5 to 30%, such as 1.0 to 20%, such as 1 to 15%, such as 1 to 10% stock solution. The reaction may be carried out in neat enzyme stock solution. Thus, in one embodiment, the solvent is cyclodextrin glucanotransferase stock solution. In one embodiment of the disclosure, the mixture comprises an amount of cyclodextrin glucanotransferase corresponding to 0.1 to 0.2% (v/v) cyclodextrin glucanotransferase stock solution, such as 0.2 to 0.3%, such as 0.3 to 0.4%, such as 0.4 to 0.5%, such as 0.5 to 0.6%, such as 0.6 to 0.7%, such as 0.7 to 0.8%, such as 0.8 to 0.9%, such as 0.9 to 1.0%, such as 1.0 to 1.5%, such as 1.5 to 2.0%, such as 2.0 to 2.5%, such as 2.5 to 3.0%, such as 3.0 to 4.0%, such as 4.0 to 5.0%, such as 5.0 to 6.0%, such as 6.0 to 7.0%, such as 7.0 to 8.0%, such as 8.0 to 9.0%, such as 9.0 to 10%, such as 10 to 15%, such as 15 to 20%, such as 20 to 25%, such as 25 to 30%, such as 30 to 40%, such as 40 to 50% cyclodextrin glucanotransferase stock solution. It is to be construed that whenever reference to cyclodextrin glucanotransferase is made here, any of the enzymes disclosed herein may be substituted for the cyclodextrin glucanotransferase. In one embodiment, the cyclodextrin glucanotransferase stock solution corresponds to or is cyclodextrin glucanotransferase having catalogue no. 970390 from Amano Enzyme Europe Limited.

In one embodiment of the disclosure, the concentration of the glucose-based compound in the solvent is 0.1 to 200 g/L, such as 0.1 to 150 g/L, such as 0.1 to 100 g/L, such as 0.2 to 90 g/L, such as 0.5 to 80 g/L, such as 0.5 to 70 g/L, such as 1 to 60 g/L, such as 1 to 50 g/L, such as 1 to 50 g/L, such as 1 to 40 g/L, such as 1 to 30 g/L, such as 1 to 25 g/L. In one embodiment, the concentration of the glucose-based compound in the solvent is 0.1 to 0.2 g/L, such as 0.2 to 0.5 g/L, such as 0.5 to 1 g/L, such as 1 to 2 g/L, such as 2 to 5 g/L, such as 5 to 10 g/L, such as 10 to 15 g/L, such as 15 to 20 g/L, such as 20 to 25 g/L, such as 25 to 30 g/L, such as 30 to 40 g/L, such as 40 to 50 g/L, such as 50 to 60 g/L, such as 60 to 70 g/L, such as 70 to 80 g/L, such as 80 to 90 g/L, such as 90 to 100 g/L.

In one embodiment, the concentration of the ion of formula (B₁₂ClₙH₁₂₋ₙ)²⁻ in the solvent is 0.1 to 300 mM, such a 0.1 to 250 mM, such as 1 to 200 mM, such as 1 to 150 mM, such as 1 to 100 mM, such as 0.2 to 90 mM, such as 0.3 to 80 mM, such as 0.4 to 70 mM, such as 0.5 to 60 mM, such a 0.6 to 50 mM, such as 0.7 to 40 mM, such as 0.8 to 30 mM, such as 0.9 to 25 mM, such as 1.0 to 20 mM, such as 1.0 to 15 mM, such as 1.0 to 10 mM. In one embodiment, the concentration of the ion of formula (B₁₂ClₙH₁₂₋ₙ)²-in the solvent is 0.1 to 0.2 mM, such as 0.2 to 0.3 mM, such as 0.3 to 0.4 mM, such as 0.4 to 0.5 mM, such as 0.5 to 0.6 mM, such as 0.6 to 0.7 mM, such as 0.7 to 0.8 mM, such as 0.8 to 0.9 mM, such as 0.9 to 1.0 mM, such as 1.0 to 1.5 mM, such as 1.5 to 2.0 mM, such as 2 to 3 mM, such as 3 to 4 mM, such as 4 to 5 mM, such as 5 to 6 mM, such as 6 to 7 mM, such as 7 to 8 mM, such as 8 to 9 mM, such as 9 to 10 mM, such as 10 to 15 mM, such as 15 to 20 mM, such as 20 to 25 mM, such as 25 to 30 mM, such as 30 to 40 mM, such as 40 to 50 mM, such as 50 to 60 mM, such as 60 to 70 mM, such as 70 to 80 mM, such as 80 to 90 mM, such as 90 to 100 mM.

### Further Method Conditions

The method of the disclosure may be carried out using the exemplary combinations of conditions as outlined below.

One embodiment provides for a method of the disclosure wherein:
a. the incubation is carried out for 30 to 60 hours, and
b. the incubation is carried out at 0 to 45° C.

One embodiment provides for a method of the disclosure wherein:
a. the incubation is carried out for 40 to 56 hours, and
b. the incubation is carried out at 20 to 30° C.

One embodiment provides for a method of the disclosure wherein:
a. the glucose-based compound is starch, a cyclodextrin such as alpha-cyclodextrin, or a modified cyclodextrin such as mono-6-deoxy alpha-cyclodextrin; and
b. the ion of formula (B₁₂ClₙH₁₂₋ₙ)²⁻ is (B₁₂Cl₁₂)²⁻, (B₁₂Cl₁₁H)²⁻,(B₁₂Cl₁₀H₂)²⁻, or (B₁₂Cl₉H₃)²⁻.

One embodiment provides for a method of the disclosure wherein:
a. the concentration of the glucose-based compound in the solvent is 1 to 50 g/L,
b. the concentration of the ion of formula (B₁₂ClₙH₁₂₋ₙ)²⁻ in the solvent is 0.5 to 25 mM, and
c. the mixture comprises an amount of cyclodextrin glucanotransferase corresponding to 0.1 to 50% (v/v) cyclodextrin glucanotransferase stock solution as disclosed herein.

One embodiment provides for a method of the disclosure wherein:
a. the concentration of the glucose-based compound in the solvent is 5 to 25 g/L,
b. the concentration of the ion of formula (B₁₂ClₙH₁₂₋ₙ)²⁻ in the solvent is 0.5 to 10 mM and
c. the mixture comprises an amount of cyclodextrin glucanotransferase corresponding to 1 to 10% (v/v) cyclodextrin glucanotransferase stock solution as disclosed herein.

One embodiment provides for a method of the disclosure wherein:
a. the incubation is carried out for 30 to 60 hours,
b. the incubation is carried out at 0 to 45° C.
c. the concentration of the glucose-based compound in the solvent is 1 to 50 g/L,
d. the concentration of the ion of formula (B₁₂ClₙH₁₂₋ₙ)²⁻ in the solvent is 0.5 to 25 mM, and
e. the mixture comprises an amount of cyclodextrin glucanotransferase corresponding to 0.1 to 50% (v/v) cyclodextrin glucanotransferase stock solution as disclosed herein.

One embodiment provides for a method of the disclosure wherein:
a. the incubation is carried out for 40 to 56 hours,
b. the incubation is carried out at 20 to 30° C.
c. the concentration of the glucose-based compound in the solvent is 5 to 25 g/L,
d. the concentration of the ion of formula (B₁₂ClₙH₁₂₋ₙ)²⁻ in the solvent is 0.5 to 10 mM, and
e. the mixture comprises an amount of cyclodextrin glucanotransferase corresponding to 1 to 10% (v/v) cyclodextrin glucanotransferase stock solution.

One embodiment provides for a method of the disclosure wherein:
a. the incubation is carried out for 30 to 60 hours,
b. the incubation is carried out at 0 to 45° C.
c. the concentration of the glucose-based compound in the solvent is 1 to 50 g/L,
d. the concentration of the ion of formula (B₁₂ClₙH₁₂₋ₙ)²⁻ in the solvent is 0.5 to 25 mM,
e. the mixture comprises an amount of cyclodextrin glucanotransferase corresponding to 0.1 to 50% (v/v) cyclodextrin glucanotransferase stock solution as disclosed herein,
f. the glucose-based compound is selected from glucose, alpha-cyclodextrin, beta-cyclodextrin, gamma-cyclodextrin, a modified alpha-cyclodextrin, a modified beta-cyclodextrin, a modified gamma-cyclodextrin, starch such as soluble starch, and a glucan, and
g. the ion of formula [B₁₂ClₙH₁₂₋ₙ]²⁻ is selected from the group consisting of (B₁₂Cl₁₂)²⁻, (B₁₂Cl₁₁H)²⁻, (B₁₂Cl₁₀H₂)²⁻, (B₁₂Cl₉H₃)²⁻, (B₁₂Cl₈H₄)²⁻, (B₁₂Cl₇H₅)²⁻, (B₁₂Cl₆H₆)²⁻, (B₁₂Cl₅H₇)²⁻, (B₁₂Cl₄H₈)²⁻, (B₁₂Cl₃H₉)²⁻, (B₁₂Cl₂H₁₀)²⁻, and (B₁₂ClH₁₁)²⁻.

One embodiment provides for a method of the disclosure wherein:
a. the incubation is carried out for 40 to 56 hours,
b. the incubation is carried out at 20 to 30° C.
c. the concentration of the glucose-based compound in the solvent is 5 to 25 g/L,
d. the concentration of the ion of formula (B₁₂ClₙH₁₂₋ₙ)²⁻ in the solvent is 0.5 to 10 mM,
e. the mixture comprises an amount of cyclodextrin glucanotransferase corresponding to 1 to 10% (v/v) cyclodextrin glucanotransferase stock solution.
f. the glucose-based compound is selected from glucose, alpha-cyclodextrin, beta-cyclodextrin, gamma-cyclodextrin, a modified alpha-cyclodextrin, a modified beta-cyclodextrin, a modified gamma-cyclodextrin, starch such as soluble starch, and a glucan, and
g. the ion of formula [B₁₂ClₙH₁₂₋ₙ]²⁻ is selected from the group consisting of (B₁₂Cl₁₂)₂, (B₁₂Cl₁₁H)²⁻, (B₁₂Cl₁₀H₂)²⁻, and (B₁₂Cl₉H₃)²⁻.

### Further Method Steps

The delta-cyclodextrin or derivative thereof obtained by the methods disclosed herein may be further modified, such as chemically modified. Thus, in one embodiment, the method of the disclosure further comprises a step of chemically modifying the delta-cyclodextrin or delta-cyclodextrin derivative obtained from the method of the disclosure.

The reaction occurring during the incubation step of the presently disclosed method can be terminated by denaturing the enzyme. Such denaturing can be carried using methods know to those of skill in the art. One such way of denaturing the enzyme is by heating it. Thus, In one embodiment, the method further comprising a step of heating the mixture to at least 70° C., such as at least 75° C., such as at least 80° C., such as at least 85° C., such as at least 90° C., such as at least 95° C., such as at least 100° C., such as at least 110° C., such as at least 120° C., such as at least 130° C. In one embodiment, said heating the mixture is for 1 to 60 min, such as 5 to 50 min, such as 5 to 40 min, such as 5 to 30 min, such as 5 to 20 min, such as for about 15 min. In one embodiment, said heating the mixture is for at least 5 min, such as at least 10 min, such as at least 15 min. In one embodiment, said heating step being carried out subsequently to the incubation step.

The delta-cyclodextrin or delta-cyclodextrin derivative may be isolated as outlined herein below.

In one embodiment, said method further comprising a step of partially evaporating the solvent from mixture. In one embodiment, said evaporating the mixture comprises removing 10 to 20% of the solvent volume, such as 20 to 30%, such as 30 to 40%, such as 40 to 50%, such as 50 to 60%, such as 60 to 70%, such as 70 to 80%, such as 80 to 90%. In one embodiment, said partially evaporating the solvent from the mixture is carried out under reduced pressure and/or by heating the mixture. In one embodiment, said partially evaporating the solvent from the mixture is carried out at atmospheric pressure and/or without heating the mixture. In one embodiment, said partially evaporating the solvent from the mixture is effective in precipitating the cyclodextrin glucanotransferase.

Because the method of the present disclosure is capable of forming delta-cyclodextrin and derivatives thereof without also forming other cyclodextrins, said delta-cyclodextrin and derivatives thereof can be isolated using precipitation and/or reprecipitation. Thus, in one embodiment, the mixture comprises a solid phase comprising cyclodextrin glucanotransferase and a liquid phase comprising delta-cyclodextrin or a delta-cyclodextrin derivative, and wherein said method further comprising a step of separating the solid phase and the liquid phase of the mixture. In one embodiment, said step of separating the solid phase comprising cyclodextrin glucanotransferase and a liquid phase comprising delta-cyclodextrin or a delta-cyclodextrin derivative is carried out using centrifuging, filtering, and/or decanting. In one embodiment, said method further comprising a step of precipitating the delta-cyclodextrin or the delta-cyclodextrin derivative from the liquid phase.

In one embodiment, the precipitation of the delta-cyclodextrin or the delta-cyclodextrin derivative comprises adding an antisolvent to the liquid phase, whereby the delta-cyclodextrin or the delta-cyclodextrin derivative precipitates. In one embodiment, the antisolvent is a protic solvent or an aprotic polar solvent. In one embodiment, the antisolvent is selected from the group consisting of acetone, ethanol, isopropanol, and acetonitrile. In one embodiment, said method further comprising a step of filtering the precipitated delta-cyclodextrin or delta-cyclodextrin derivative.

In one embodiment, the method further comprises a step of:
a. dissolving in water and/or making a slurry in water of the delta-cyclodextrin or delta-cyclodextrin derivative,
b. adding an antisolvent, such as a protic solvent or an aprotic polar solvent, such as acetone, ethanol, isopropanol, or acetonitrile, thereby precipitating the delta-cyclodextrin or delta-cyclodextrin derivative, and
c. filtering the precipitated delta-cyclodextrin or delta-cyclodextrin derivative.

### Exemplary Embodiments

In one embodiment, the method comprises the steps carried out in the order:
a. mixing:
   v. a glucose-based compound,
   vi. a cyclodextrin glucanotransferase,
   vii. an ion of formula (B₁₂ClₙH₁₂₋ₙ)²⁻, wherein n is 1 to 12, and
   viii. a solvent to form a mixture;
b. incubating the mixture by heating;
c. partially evaporating the solvent from mixture to form a solid phase and a liquid phase;
d. separating the solid phase and the liquid phase of the mixture;
e. adding antisolvent to said liquid phase to precipitate the delta-cyclodextrin or the delta-cyclodextrin derivative; and
f. filtering the precipitated delta-cyclodextrin or the delta-cyclodextrin derivative.

The template ion employ in the method of the disclose can be recovered and reused to carry out the method of the disclosure again. In one embodiment, the method further comprises recovering the ion of formula (B₁₂ClₙH₁₂₋ₙ)²⁻, said recovery comprising:
a. obtaining the supernatant from the precipitation step of the delta-cyclodextrin or the delta-cyclodextrin derivative,
b. evaporating antisolvent under reduced pressure,
c. adding hydrochloric acid, such as 37% hydrochloric acid to obtain a pH between 1 and 4, such as between 1.5 and 2.5,
d. adding a base, such as an amine base, such as an alkyl amine base, such as a trialkyl amine compound, such as triethylamine to precipitate the ion of formula (B₁₂ClₙH₁₂₋ₙ)²⁻ as a salt of said base, and
e. filtering the precipitate,
thereby obtaining the ion of formula (B₁₂ClₙH₁₂₋ₙ)²⁻ as a salt of said base.

One embodiment of the present disclosure provides for a method of isolating an ion of formula (B₁₂ClₙH₁₂₋ₙ)²⁻ from a mixture of components, said isolation comprising:
a. obtaining an aqueous liquid phase comprising the ion of formula (B₁₂ClₙH₁₂₋ₙ)²⁻,
b. adding hydrochloric acid, such as 37% hydrochloric acid to obtain a pH between 1 and 4, such as between 1.5 and 2.5,
c. adding a base, such as an amine base, such as an alkyl amine base, such as a trialkyl amine compound, such as triethylamine to precipitate the ion of formula (B₁₂ClₙH₁₂₋ₙ)²⁻ as a salt of said base, and
d. filtering the precipitate,
thereby obtaining the ion of formula (B₁₂ClₙH₁₂₋ₙ)²⁻ as a salt of said base.

One embodiment of the disclosure provides for a method for producing delta-cyclodextrin or a delta-cyclodextrin derivative, said method comprising in order the steps of:
a. mixing:
   i. a glucose-based compound,
   ii. a cyclodextrin glucanotransferase,
   iii. an ion of formula (B₁₂ClₙH₁₂₋ₙ)²⁻, wherein n is 1 to 12, and
   iv. a solvent
   to form a mixture;
b. incubating the mixture by heating it to at least 70° C. for at least 5 min;
c. partially evaporating the solvent from mixture, whereby the cyclodextrin glucanotransferase is precipitated, thereby forming a solid phase and a liquid phase;
d. separating the solid phase and the liquid phase of the mixture;
e. adding antisolvent such as a protic solvent or an aprotic polar solvent to said liquid phase, whereby the delta-cyclodextrin or the delta-cyclodextrin derivative precipitates; and
f. filtering the precipitated delta-cyclodextrin or delta-cyclodextrin derivative;
thereby obtaining the delta-cyclodextrin or the delta-cyclodextrin derivative.

One embodiment of the present disclosure provides for a delta-cyclodextrin or a delta-cyclodextrin derivative obtained from the method as disclosed herein.

One embodiment of the disclosure provides for a composition comprising:
a. a glucose-based compound as disclosed herein,
b. an enzyme such as a cyclodextrin glucanotransferase as disclosed herein, and
c. an ion of formula (B₁₂ClₙH₁₂₋ₙ)²⁻ as disclosed herein.

Such a composition is useful in the disclosed methods.

One embodiment of the present disclosure provides for a kit of parts comprising:
a. a glucose-based compound as disclosed herein,
b. a enzyme such as a cyclodextrin glucanotransferase as disclosed herein, and
c. a salt comprising an ion of formula (B₁₂ClₙH₁₂₋ₙ)²⁻ as disclosed herein.

### EXAMPLES

### Example 1: Procedure for Analytical Scale Treatment of CD6 with CGTase in the Absence of Template

### Materials and Methods

All solutions were prepared in buffered water (50 mM sodium phosphate at pH 7.5). 50 μL of a alpha-CD solution (20 mg/mL) were added to a 0.5 mL vial at 25° C. then buffer (43.5 μL) was added to a total volume of 93.5 μL. The reaction was initiated by adding CGTase stock solution* (6.5 μL) resulting in a 100 μL CD6 solution (10 mg/mL) (*: the commercial supplier has not disclosed the concentration of CGTase in the stock solution). The CGTase was purchased from Amano Enzyme Europe Limited, Item no. 970390. The reaction was monitored at various time points. Aliquots for analysis (3 μL) were removed and the enzymatic reaction was stopped by immediate addition to 90 μL of a 1% (v/v) solution of trifluoroacetic acid in acetonitrile/water (3:1). The samples were analysed using high-performance liquid chromatography with evaporative light scattering detection (HPLC-ELS). Separation was performed using gradient elution on a HILIC-type column.

### Results

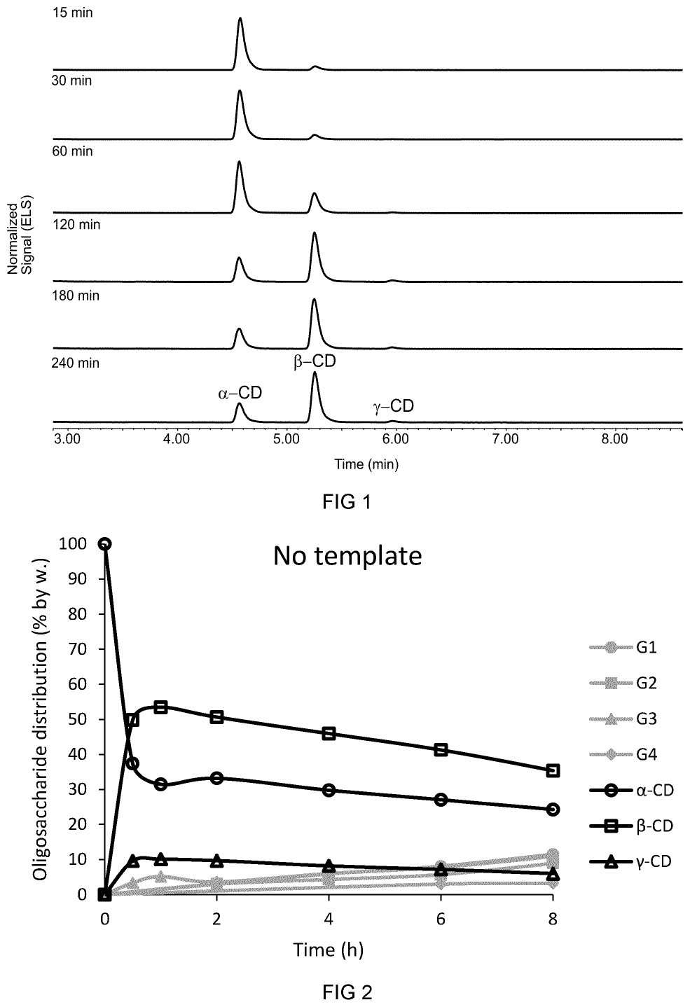

The HPLC-ELS chromatograms show that CD6 is transformed into a mixture of CD6, CD7 and CD8 (FIG. 1) as well as a small amount of linear alpha-1,4-glucans. The areas under each peak can be converted to concentrations using calibration curves made with genuine samples of each of the relevant species, allowing the concentration of CDs and linear alpha-1,4-glucans to be quantified. The evolution of the reaction mixture over time is plotted in FIG. 2.

### Conclusion

When CD6 (10 mg/mL) is treated with the commercially available enzyme cyclodextrin glucanotransferase (CGTase from Amano Enzyme) in phosphate buffer at pH 7.5 at 25° C., it is transformed into a mixture of mainly CD6, CD7 and CD8 (FIGS. 1 and 2).

### Example 2: Procedure for Analytical Scale Synthesis of CD9 from CD6 Using Na₂B₁₂Cl₁₂ as a Template

### Materials and Methods

All solutions were prepared in buffered water (50 mM sodium phosphate at pH 7.5). 50 μL of an alpha-CD solution (20 mg/mL) and 8 μL of a 50 mM Na₂B₁₂Cl₁₂ solution were added to a 0.5 mL vial at 25° C. Then buffer (35.5 μL) was added to a total volume of 93.5 μL. The reaction was initiated by adding CGTase stock solution (6.5 μL) resulting in a 100 μL reaction volume with 10 mg/mL alpha-CD and 4 mM Na₂B₁₂Cl₁₂ template. The reaction was monitored at various time points. Aliquots for analysis (3 μL) were removed and the enzymatic reaction was stopped by immediate addition of this aliquot to 90 μL of a 1% (v/v) solution of trifluoroacetic acid in acetonitrile/water (3:1). The reaction progress and CD mixture composition was analysed using high-performance liquid chromatography with evaporative light scattering detection (HPLC-ELS). Separation was performed using gradient elution on a HILIC-type column. The same procedure was also carried out separately at 5° C. and at 40° C. The reaction was also carried out at 25° C. with final Na₂B₁₂Cl₁₂ concentrations of 2 mM or 30 mM by appropriate adjustments of the volumes of the Na₂B₁₂Cl₁₂ stock solution and buffer in the above-mentioned procedure.

### Results

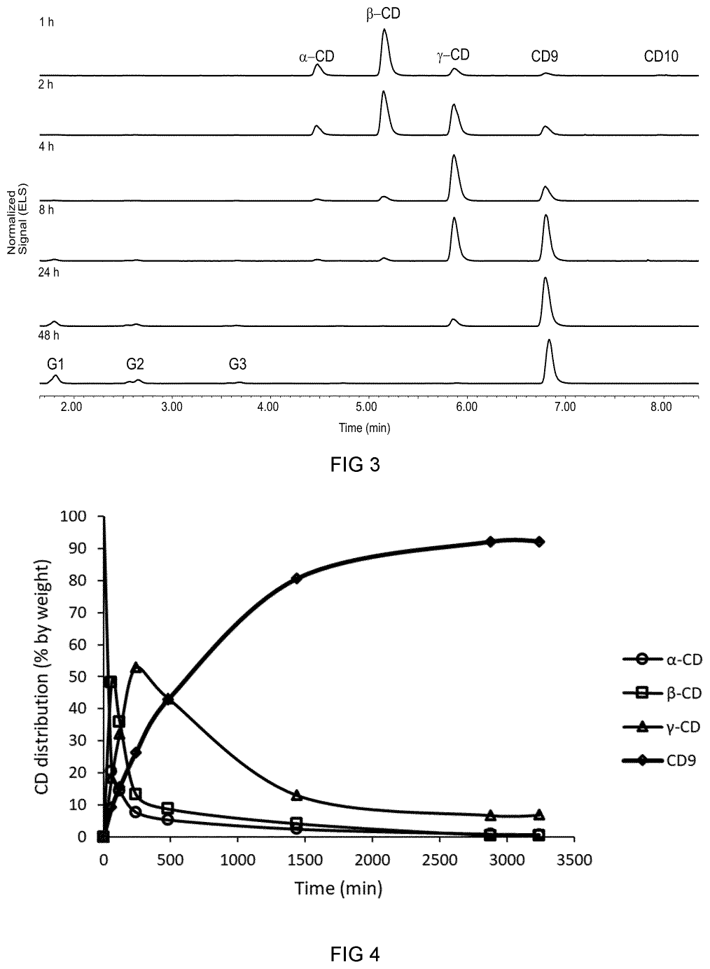

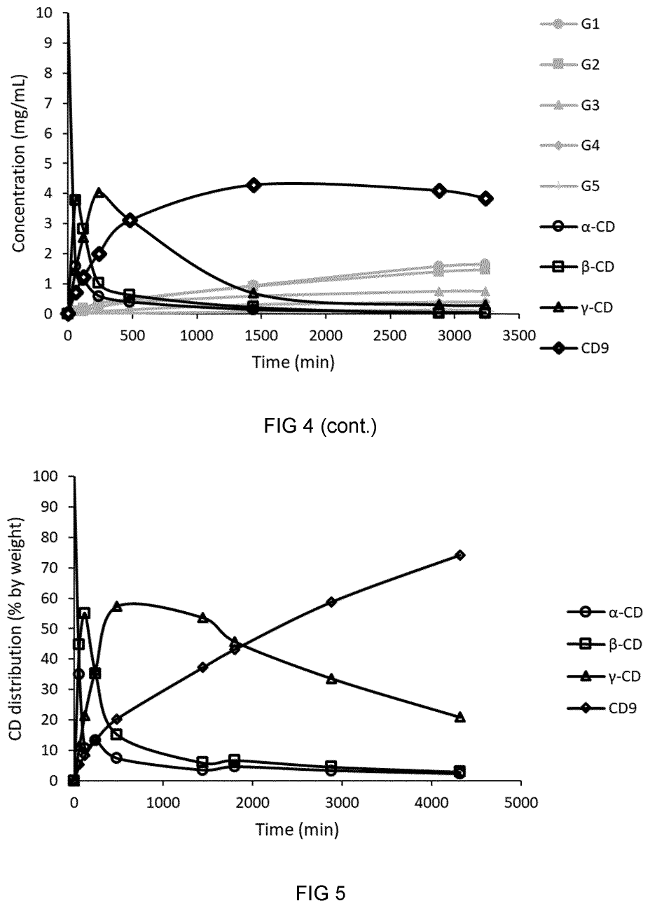

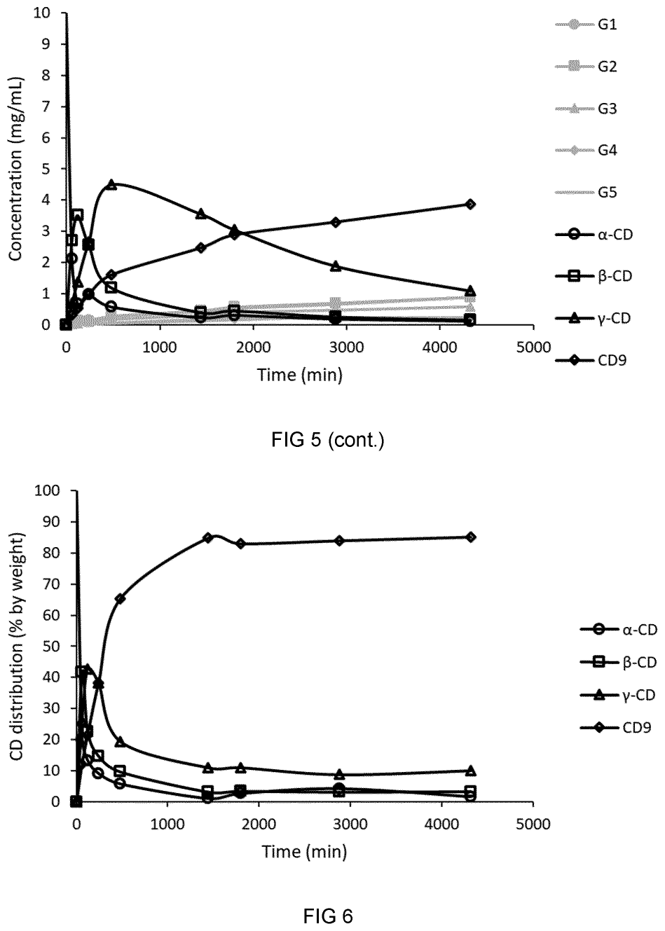

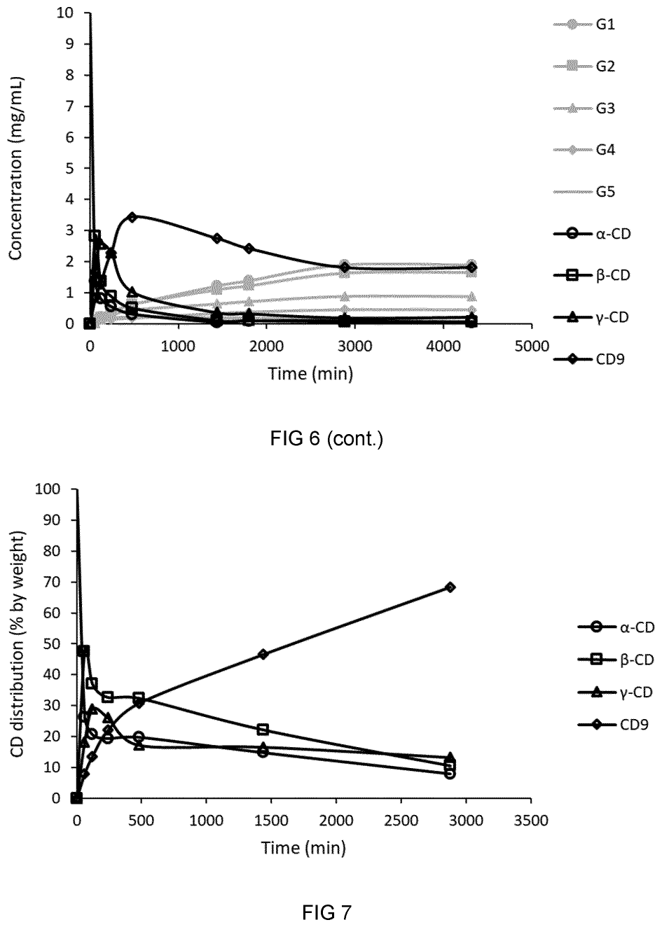

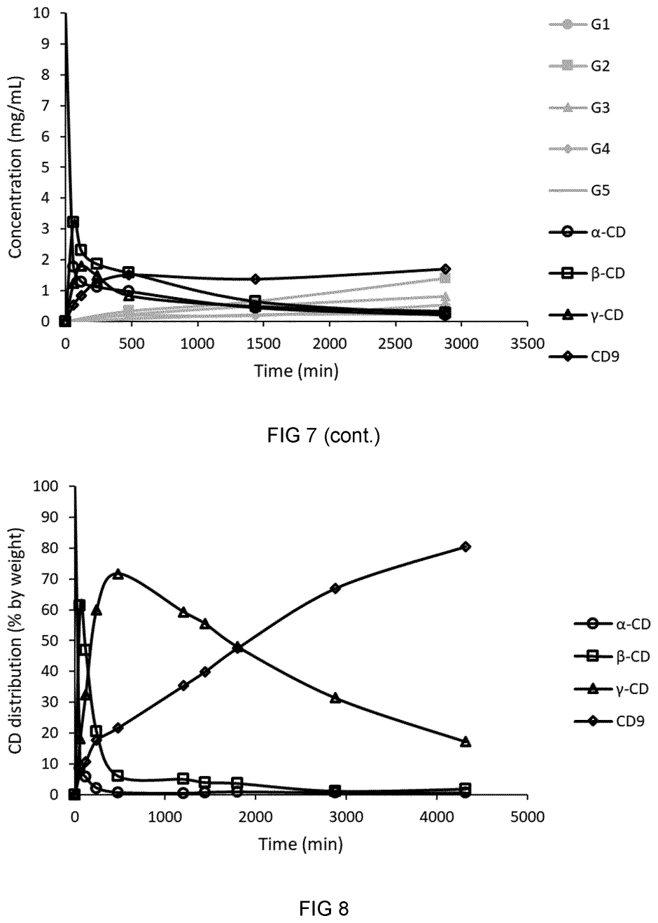

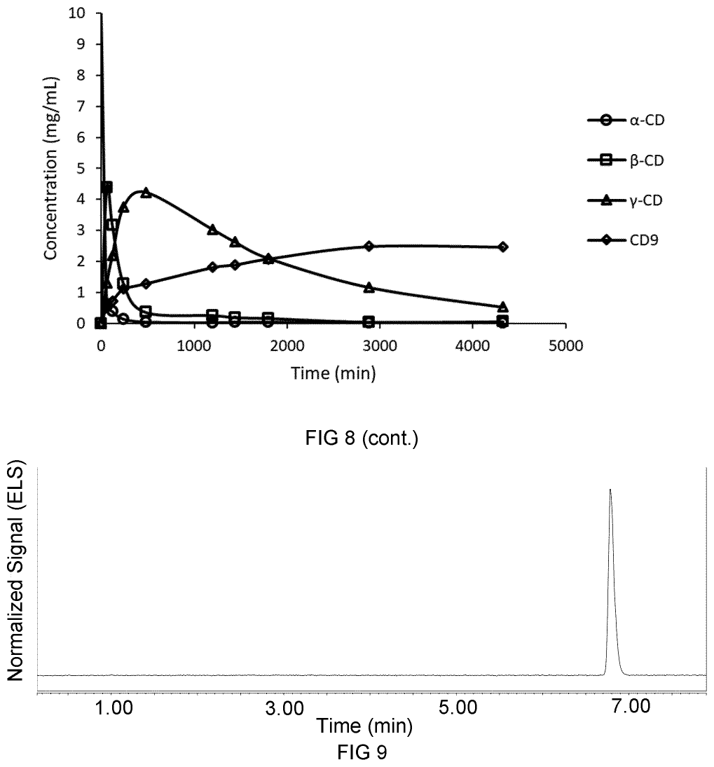

For the reaction at 25° C. with 4 mM Na₂B₁₂Cl₁₂, the HPLC-ELS chromatograms show that CD6 is transformed into a mixture consisting of primarily CD9 as well as a small amount of linear alpha-1,4-glucans (FIG. 3). The areas under each peak were converted to concentrations using calibration curves made with genuine samples of each of the relevant species, allowing the concentration of CDs and linear alpha-1,4-glucans to be quantified over time. After 48 hours of reaction, CD9 was produced as the major product in the reaction mixture. It was present at a concentration of 4.0 mg/mL and was produced with high selectivity, as it accounted for more than 90% of the total CD composition (FIG. 4). At 5° C., the reaction progressed more slowly, but also produced primarily CD9. CD9 was generated a concentration of 3.3 mg/mL with a selectivity of 59% after 48 hours. Selectivity for CD9 improved as the reaction progressed, with about 20% CD8 present out of the CD composition) after 3 days of reaction (FIG. 5). At 40° C., the formation of CD9 was faster and a maximum concentration of CD9 (3.4 mg/mL) was generated after 8 hours. At this time point 65% selectivity for CD9 was observed. A longer reaction time gave an increase in selectivity for CD9 (up to 85% at best) with a somewhat lower overall yield (FIG. 6). In the reaction with 2 mM template at 25° C., CD9 was again formed as the major product with a maximum concentration of 1.5 mg/mL obtained within 8 hours. After 8 hours, CD9 accounted for 31% of the CD composition and after 2 days, CD6, CD7 and CD8 made up 32% of the CD composition. In the reaction with 30 mM template at 25° C., a maximum concentration of CD9 of 2.5 mg/mL was obtained after 2 days, while CD9 accounted for 67% of the CD composition at this time and after three days CD6, CD7 and CD8 made up 29% of the CD composition (FIG. 8).

### Conclusion

When CD6 (10 mg/mL) is treated with CGTase in phosphate buffer at pH 7.5 at 25° C. in the presence of Na₂B₁₂Cl₁₂ (4 mM), the major product generated is CD9 (FIGS. 3 and 4). When higher or lower temperature (4° C. or 40° C.) or different template concentrations (2 mM or 30 mM) were employed, CD9 was still the major product.

### Example 3: Procedure for Large Scale Synthesis of CD9

### Materials and Methods

CD6 (10.00 g, 10.03 mmol) and Na₂B₁₂Cl₁₂ (3.00 g, 5 mmol) were dissolved in MilliQ water (900 mL) adjusted to pH 7.5 with 1 M HCl. The mixture was transferred to a 1-liter volumetric flask. A solution of CGTase (25 mL) was then added to start the reaction and MilliQ water was added until the 1-L mark. The reaction mixture was transferred to a 2-liter round bottomed flask and placed in a water bath at 30° C. for 42 hours, after which the reaction was stopped by heating to boiling for 15 minutes. The reaction mixture was concentrated to 0.2 L in vacuo, then centrifuged and the supernatant decanted, leaving behind precipitated enzyme. CD9 was then precipitated by the addition of acetone (1.0 L), and isolated by filtration. The white solids collected were then dissolved in water (100 mL) and precipitated with acetone (500 mL), followed by filtration to isolate the solids. This process was repeated 2 times, after which the white solids were dried in vacuo.

### Results

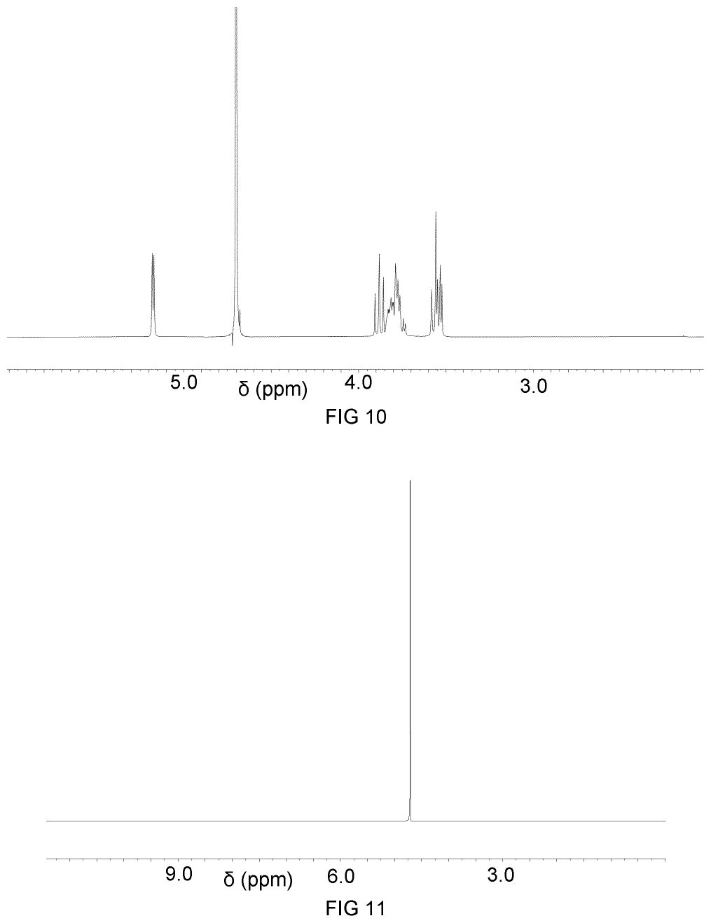

CD9 was isolated as a white solid (4.67 g, 47% yield) with a purity of >99% (by HPLC-ELSD, FIG. 9). Even higher purity can be obtained by additional reprecipitation, as shown by ¹H NMR spectroscopy (FIG. 10). In a modification of the above-mentioned procedure, it was found that CD9 could also be isolated by precipitation from the above-mentioned aqueous solution in the same fashion by substituting the acetone for other organic solvents, e.g. ethanol, isopropanol, acetonitrile.

### Conclusion

High-purity CD9 was obtained by treatment of CD6 with CGTase in water in the presence of Na₂B₁₂Cl₁₂, and subsequently isolated by precipitation from aqueous solution using an organic solvent.

### Example 4: Recovery of the Na₂B₁₂Cl₁₂ Template

### Materials and Methods

The filtrate and washings from the isolation procedure of CD9 were combined and concentrated in vacuo to remove acetone and give a total volume of 200 mL. Then HCl (37%) was added to reach pH 2, followed by addition of triethylamine in small portions with stirring until no more gas evolved. The white precipitate that formed was collected by filtration and washed several times with cold water, then dried in vacuo. This Et₃NHB₁₂Cl₁₂ was converted to the sodium salt following the procedure reported by Geis et al.

### Results

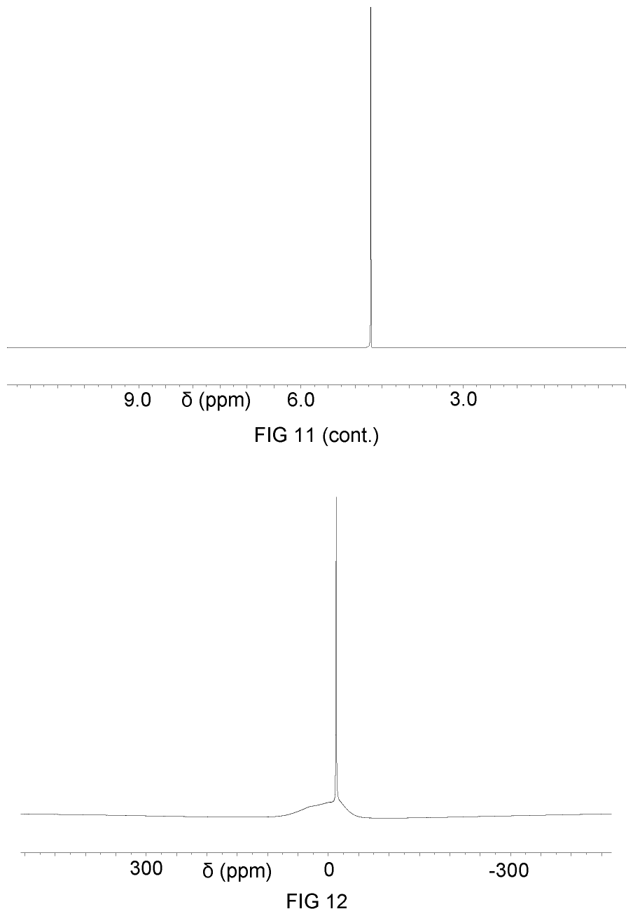

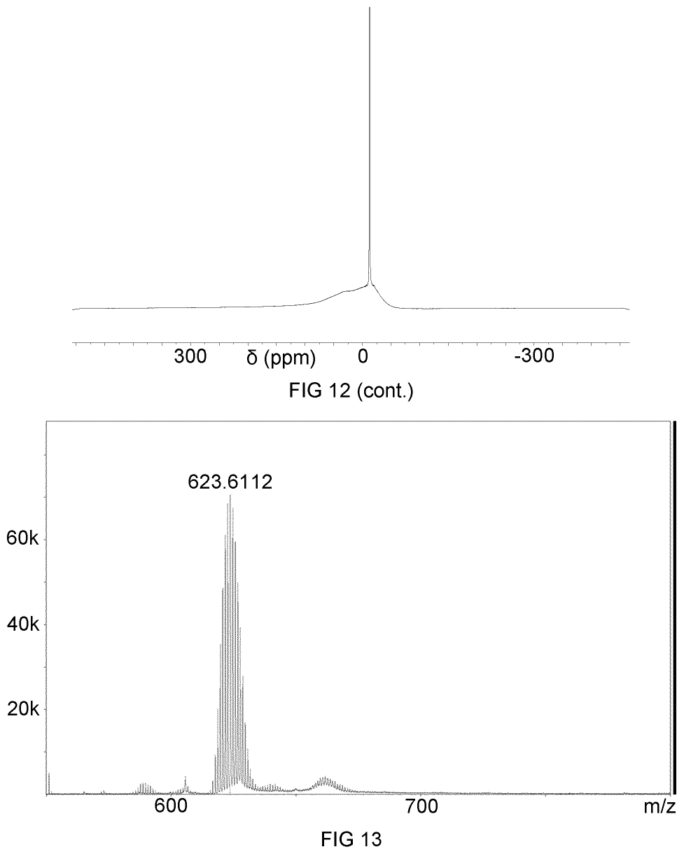

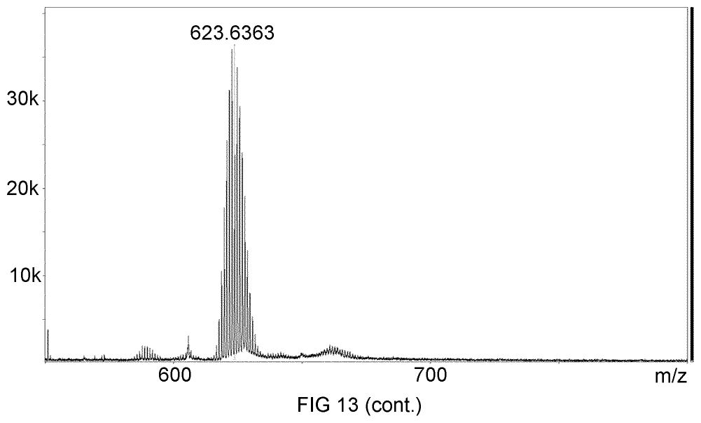

The Na₂B₁₂Cl₁₂ template was recovered from the reaction mixture in quantitative yield. The purity of the recovered template was confirmed to be at least at the same level as before the recovery by ¹H and ¹¹B NMR spectroscopy (FIGS. 11 and 12) and MALDI mass spectrometry (FIG. 13).

The recycled template was successfully used to produce more delta-cyclodextrin.

### Conclusion

The Na₂B₁₂Cl₁₂ template can be re-isolated directly from the above-mentioned reaction mixture allowing for its reuse.

### Example 5: Procedure for Synthesis of Modified CD9s

### Materials and Methods

To a solution of Na₂B₁₂Cl₁₂ (50 mM) and mono-(6-deoxy)-CD6 (10 mg/mL) in buffered water (50 mM sodium phosphate at pH 7.5) was added CGTase stock solution (50 μL per mL). The reaction was monitored at various time points. Aliquots for analysis (5 μL) were removed and the enzymatic reaction was stopped by immediate addition of this aliquot to 80 μL of a 1% (v/v) solution of trifluoroacetic acid in acetonitrile/water (3:1). The reaction progress and CD mixture composition was analysed using matrix-assisted laser desorption/ionisation time-of-flight mass spectrometry (MALDI-TOF-MS) and high-performance liquid chromatography with evaporative light scattering detection (HPLC-ELS), where separation was performed using gradient elution on a HILIC-type column.

### Results

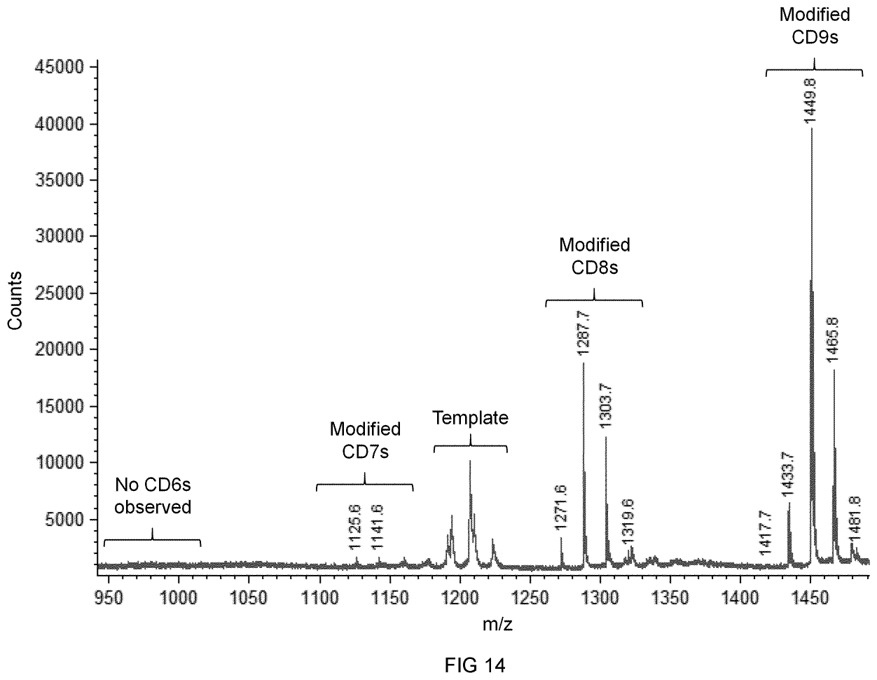

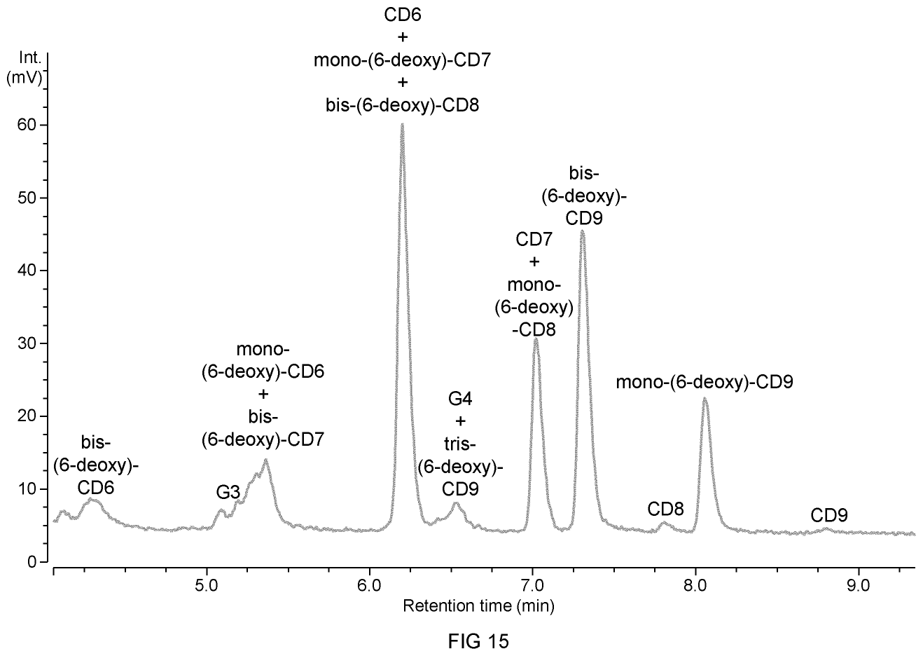

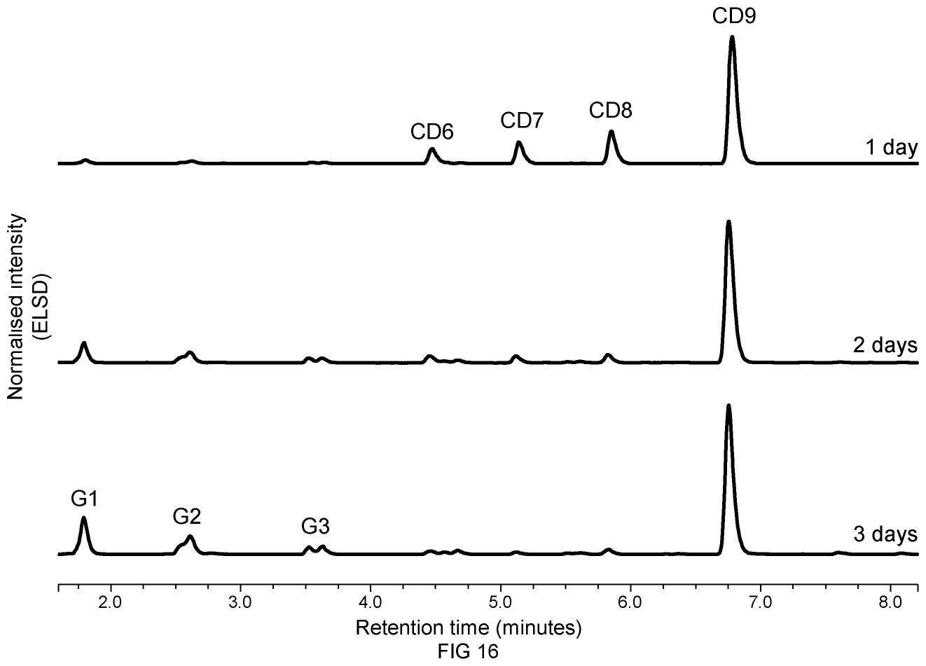

After 20 days of reaction, the obtained mass spectrum indicates formation of primarily CD9 (m/z 1481.4 expected for [M+Na]⁺) and 6-deoxy-CD9s (m/z 1465.5, 1449.5, 1433.5 and 1417.5 expected for [M+Na]⁺ for mono-, bis-, tris- and tetrakis-(6-deoxy)-CD9, respectively) (FIG. 14). CD8 (m/z 1319.4 expected for [M+Na]+), 6-deoxy-CD8s (m/z 1303.4, 1287.4 and 1271.4 expected for [M+Na]⁺ for mono-, bis- and tris-(6-deoxy)-CD8, respectively) and 6-deoxy-CD7s (m/z 1141.4 and 1125.4 expected for [M+Na]⁺ for mono- and bis-(6-deoxy)-CD7, respectively) were also observed. These results were confirmed by the HPLC-ELS chromatogram (FIG. 15), in which the compounds responsible for peaks at different retention times were identified either by comparison to genuine samples (all unmodified sugars) or by fractional collection followed by MALDI-TOF-MS (all the modified CDs).

### Conclusion

Use of Na₂B₁₂Cl₁₂ as a template also favours the synthesis of modified CD9s.

### Example 6: Procedure for the Production of CD9 from Starch

### Materials and Methods

Soluble starch (from commercial supplier Sigma-Aldrich, product no. S9765) (2.00 g) was dissolved in water (80 mL, by heating in microwave for 4×1 minute and then heating in an oil bath to boiling temperature for 20 minutes with stirring). To this solution was added Na₂B₁₂Cl₁₂ (0.300 g, 0.50 mmol) and the pH was adjusted to 7.5 with 1 M HCl. The mixture was transferred to a 100 mL volumetric flask. CGTase stock solution (2.5 mL) was then added to start the reaction and MilliQ water was used to adjust the volume to 100 mL resulting in a starch concentration of 20 mg/mL. The reaction was monitored at various time points. Aliquots for analysis (3 μL) were removed and the enzymatic reaction was stopped by immediate addition of this aliquot to 90 μL of a 1% (v/v) solution of trifluoroacetic acid in acetonitrile/water (3:1). The reaction progress and CD mixture composition was analysed using high-performance liquid chromatography with evaporative light scattering detection (HPLC-ELS). Separation was performed using gradient elution on a HILIC-type column.

### Results

Analysis of the reaction mixture over the first three days showed production of several CDs as well as some short linear alpha-1,4-glucans (FIG. 16). The main product was found to be CD9, similar to what was found in the reactions started from CD6 (see above).

### Conclusion

Treatment of starch with CGTase in the presence of Na₂B₁₂Cl₁₂ results in formation of primarily CD9.

### Example 7: Procedure for the Production of CD9 Using Na₂B₁₂HₓCl₁₂₋ₓ Templates

### Materials and Methods

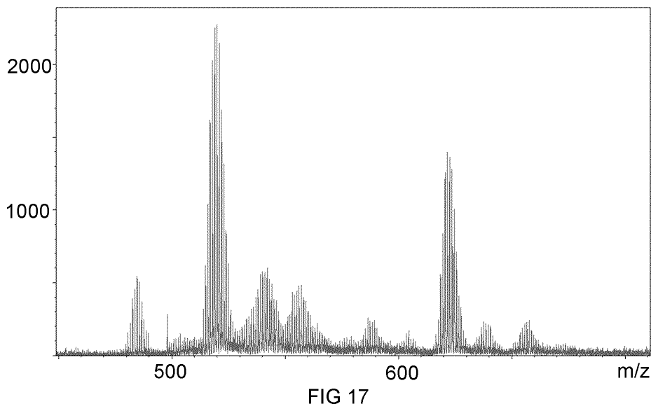

To a solution of partially chlorinated closo-dodecaborate Na₂B₁₂HₓCl₁₂₋ₓ (5 mM, assuming an average MW of 567 g/mol, see MALDI-MS in FIG. 17) and CD6 (10 mg/mL) in buffered water (50 mM sodium phosphate at pH 7.5) was added CGTase stock solution (50 μL per mL). The reaction was monitored at various time points. Aliquots for analysis (3 μL) were removed and the enzymatic reaction was stopped by immediate addition of this aliquot to 90 μL of a 1% (v/v) solution of trifluoroacetic acid in acetonitrile/water (3:1). The reaction progress and CD mixture composition was analysed using high-performance liquid chromatography with evaporative light scattering detection (HPLC-ELS). Separation was performed using gradient elution on a HILIC-type column.

### Results

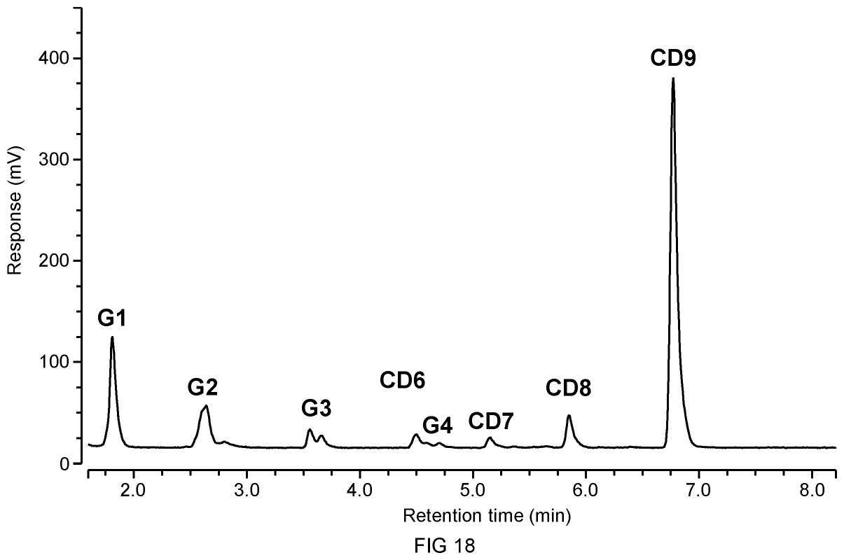

Analysis of the reaction mixture after 24 hours showed production of CD6, CD7, CD8 and CD9 as well as some short linear alpha-1,4-glucans (FIG. 18). The main product was found to be CD9.

### Conclusion

CD9 is also the major product in enzymatic reactions employing templates that are similar to Na₂B₁₂Cl₁₂.

### REFERENCES

Cyclodextrins used as excipients, European Medicines Agency, Report published in support of the “Questions and answers on cyclodextrins used as excipients in medicinal products for human use' EMA/CHMP/495747/2013.
V. Geis, K. Guttsche, C. Knapp, H. Scherer, R. Uzun, Dalton Trans. 2009, 2687.

1. A method for producing delta-cyclodextrin or a delta-cyclodextrin derivative, said method comprising mixing:
a. a glucose-based compound,
b. a cyclodextrin glucanotransferase,
c. an ion of formula (B₁₂ClₙH₁₂₋ₙ)²⁻, wherein n is 1 to 12, and
d. a solvent
to form a mixture, and
incubating the mixture,
thereby obtaining the delta-cyclodextrin or a delta-cyclodextrin derivative.

2. The method according to claim 1, wherein the delta-cyclodextrin or the delta-cyclodextrin derivative has the structure of formula I: 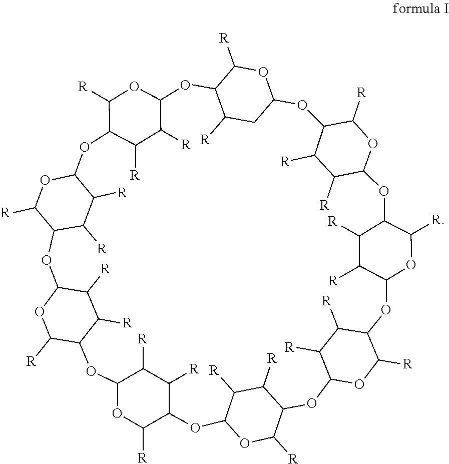
or a salt thereof,
wherein each R is independently selected from the group consisting of CH₂OR^{a}, CH₃, CH₂F, CH₂Cl, CH₂Br, CH₂I, CH₂N₃, CH₂CN, CH₂R^{a}, CH₂N(R^{a})₂, CH₂SR^{a}, CH₂Si(R^{a})₃, CH₂CHO, CH₂COOH, CH₂COOR^{a}, CH₂CON(R^{a})₂, CH₂CHNR^{a}, OR^{a}, H, F, Cl, Br, I, N₃, CN, R^{a}, N(R^{a})₂, SR^{a}, Si(R^{a})₃, CHO, COOH, COOR^{a}, CON(R^{a})₂, and CHNR^{a}, and
wherein each R^{a} is independently selected from the group consisting of H, alkyl, substituted alkyl, alkenyl, substituted alkenyl, alkynyl, substituted alkynyl, acyl, substituted acyl, aryl, substituted aryl, benzyl, and substituted benzyl.

3. The method according to any one of the preceding claims wherein the delta-cyclodextrin or the delta-cyclodextrin derivative has the structure of formula Ia:

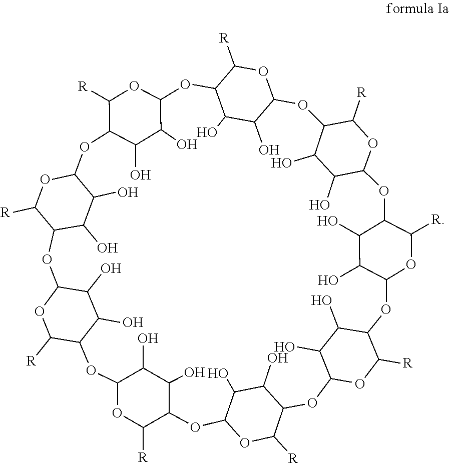

4. The method according to any one of the preceding claims, wherein the delta-cyclodextrin or the delta-cyclodextrin derivative has the structure of formula Ib: 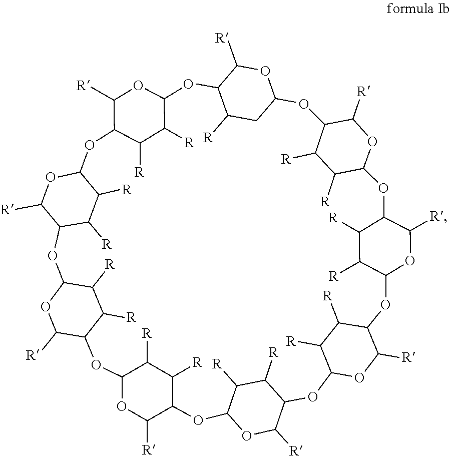
wherein each R′ is independently selected from the group consisting of CH₂OR^{a}, CH₃, CH₂F, CH₂Cl, CH₂Br, CH₂I, CH₂N₃, CH₂CN, CH₂R^{a}, CH₂N(R^{a})₂, CH₂SR^{a}, CH₂Si(R^{a})₃, CH₂CHO, CH₂COOH, CH₂COOR^{a}, CH₂CON(R^{a})₂, CH₂CHNR^{a}, OR^{a}, H, F, Cl, Br, I, N₃, CN, R^{a}, N(R^{a})₂, SR^{a}, Si(R^{a})₃, CHO, COOH, COOR^{a}, CON(R^{a})₂, and CHNR^{a},
wherein each R is independently selected from the group consisting of OR^{a}, H, F, Cl, Br, I, N₃, CN, R^{a}, N(R^{a})₂, SR^{a}, Si(R^{a})₃, CHO, COOH, COOR^{a}, CON(R^{a})₂, and CHNR^{a}, and
wherein each R^{a} is independently selected from the group consisting of H, alkyl, substituted alkyl, alkenyl, substituted alkenyl, alkynyl, substituted alkynyl, acyl, substituted acyl, aryl, substituted aryl, benzyl, and substituted benzyl.

5. The method according to any one of the preceding claims, wherein the delta-cyclodextrin or the delta-cyclodextrin derivative has the structure of formula Ic:

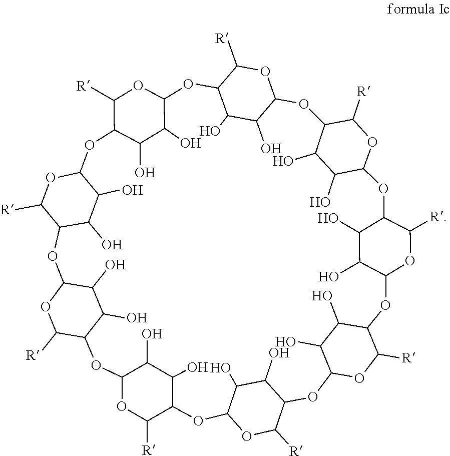

6. The method according to any one of the preceding claims, wherein the glucose-based compound is linear or cyclic.

7. The method according to any one of the preceding claims, wherein the glucose-based compound has the structure of formula II: 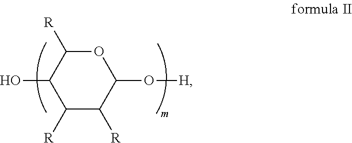
wherein each R is independently selected from the group consisting of CH₂OR^{a}, CH₃, CH₂F, CH₂Cl, CH₂Br, CH₂I, CH₂N₃, CH₂CN, CH₂R^{a}, CH₂N(R^{a})₂, CH₂SR^{a}, CH₂Si(R^{a})₃, CH₂CHO, CH₂COOH, CH₂COOR^{a}, CH₂CON(R^{a})₂, CH₂CHNR^{a}, OR^{a}, H, F, Cl, Br, I, N₃, CN, R^{a}, N(R^{a})₂, SR^{a}, Si(R^{a})₃, CHO, COOH, COOR^{a}, CON(R^{a})₂, and CHNR^{a},
wherein each R^{a} is independently selected from the group consisting of H, alkyl, substituted alkyl, alkenyl, substituted alkenyl, alkynyl, substituted alkynyl, acyl, substituted acyl, aryl, substituted aryl, benzyl, and substituted benzyl, and wherein m is an integer 2 or more.

8. The method according to any one of the preceding claims, wherein the glucose-based compound has the structure of formula IIa:

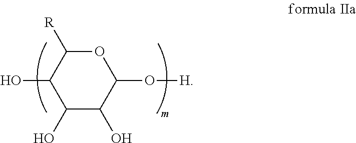

9. The method according to any one of the preceding claims, wherein the glucose-based compound has the structure of formula IIb: 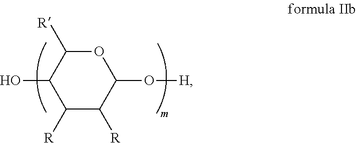
wherein each R′ is independently selected from the group consisting of CH₂OR^{a}, CH₃, CH₂F, CH₂Cl, CH₂Br, CH₂I, CH₂N₃, CH₂CN, CH₂R^{a}, CH₂N(R^{a})₂, CH₂SR^{a}, CH₂Si(R^{a})₃, CH₂CHO, CH₂COOH, CH₂COOR^{a}, CH₂CON(R^{a})₂, CH₂CHNR^{a}, OR^{a}, H, F, Cl, Br, I, N₃, CN, R^{a}, N(R^{a})₂, SR^{a}, Si(R^{a})₃, CHO, COOH, COOR^{a}, CON(R^{a})₂, and CHNR^{a},
wherein each R is independently selected from the group consisting of OR^{a}, H, F, Cl, Br, I, N₃, CN, R^{a}, N(R^{a})₂, SR^{a}, and Si(R^{a})₃,
wherein each R^{a} is independently selected from the group consisting of H, alkyl, substituted alkyl, alkenyl, substituted alkenyl, alkynyl, substituted alkynyl, acyl, substituted acyl, aryl, substituted aryl, benzyl, and substituted benzyl, and wherein m is an integer 2 or more.

10. The method according to any one of the preceding claims, wherein the glucose-based compound has the structure of formula IIc:

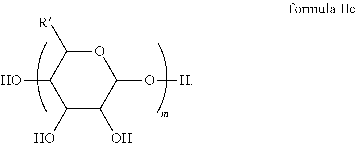

11. The method according to any one of the preceding claims, wherein the glucose-based compound has the structure of formula III: 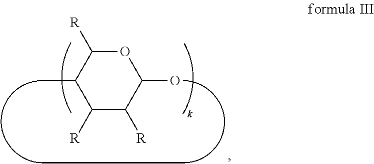
wherein each R is independently selected from the group consisting of CH₂OR^{a}, CH₃, CH₂F, CH₂Cl, CH₂Br, CH₂I, CH₂N₃, CH₂CN, CH₂R^{a}, CH₂N(R^{a})₂, CH₂SR^{a}, CH₂Si(R^{a})₃, CH₂CHO, CH₂COOH, CH₂COOR^{a}, CH₂CON(R^{a})₂, CH₂CHNR^{a}, OR^{a}, H, F, Cl, Br, I, N₃, CN, R^{a}, N(R^{a})₂, SR^{a}, Si(R^{a})₃, CHO, COOH, COOR^{a}, CON(R^{a})₂, and CHNR^{a},
wherein each R^{a} is independently selected from the group consisting of H, alkyl, substituted alkyl, alkenyl, substituted alkenyl, alkynyl, substituted alkynyl, acyl, substituted acyl, aryl, substituted aryl, benzyl, and substituted benzyl, and wherein k is an integer 6, 7, 8, 10, or more than 10.

12. The method according to any one of the preceding claims, wherein the glucose-based compound has the structure of formula IIIa:

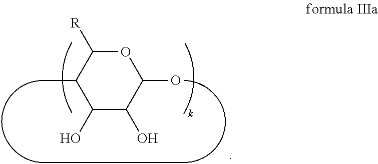

13. The method according to any one of the preceding claims, wherein the glucose-based compound has the structure of formula IIIb: 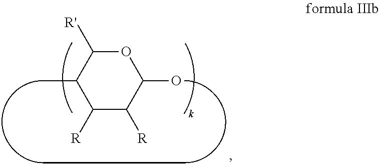
wherein each R′ is independently selected from the group consisting of CH₂OR^{a}, CH₃, CH₂F, CH₂Cl, CH₂Br, CH₂I, CH₂N₃, CH₂CN, CH₂R^{a}, CH₂N(R^{a})₂, CH₂SR^{a}, CH₂Si(R^{a})₃, CH₂CHO, CH₂COOH, CH₂COOR^{a}, CH₂CON(R^{a})₂, CH₂CHNR^{a}, OR^{a}, H, F, Cl, Br, I, N₃, CN, R^{a}, N(R^{a})₂, SR^{a}, Si(R^{a})₃, CHO, COOH, COOR^{a}, CON(R^{a})₂, and CHNR^{a},
wherein each R is independently selected from the group consisting of OR^{a}, H, F, Cl, Br, I, N₃, CN, R^{a}, N(R^{a})₂, SR^{a}, and Si(R^{a})₃,
wherein each R^{a} is independently selected from the group consisting of H, alkyl, substituted alkyl, alkenyl, substituted alkenyl, alkynyl, substituted alkynyl, acyl, substituted acyl, aryl, substituted aryl, benzyl, and substituted benzyl, and wherein k is an integer 6, 7, 8, 10, or more than 10.

14. The method according to any one of the preceding claims, wherein the glucose-based compound has the structure of formula IIIc:

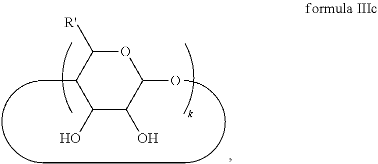

15. The method according to any one of the preceding claims, wherein the mixture further comprises a second glucose-based compound having the structure for formula IV: 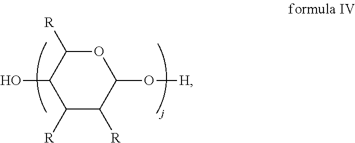
wherein each R is independently selected from the group consisting of CH₂OR^{a}, CH₃, CH₂F, CH₂Cl, CH₂Br, CH₂I, CH₂N₃, CH₂CN, CH₂R^{a}, CH₂N(R^{a})₂, CH₂SR^{a}, CH₂Si(R^{a})₃, CH₂CHO, CH₂COOH, CH₂COOR^{a}, CH₂CON(R^{a})₂, CH₂CHNR^{a}, OR^{a}, H, F, Cl, Br, I, N₃, CN, R^{a}, N(R^{a})₂, SR^{a}, Si(R^{a})₃, CHO, COOH, COOR^{a}, CON(R^{a})₂, and CHNR^{a},
wherein each R^{a} is independently selected from the group consisting of H, alkyl, substituted alkyl, alkenyl, substituted alkenyl, alkynyl, substituted alkynyl, acyl, substituted acyl, aryl, substituted aryl, benzyl, and substituted benzyl, and wherein j is an integer 1 or more.

16. The method according to any one of the preceding claims, wherein the second glucose-based compound has the structure of formula IVa:

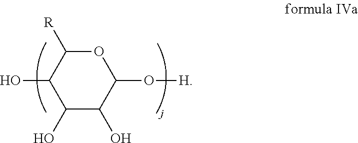

17. The method according to any one of the preceding claims, wherein the second glucose-based compound has the structure of formula IVb: 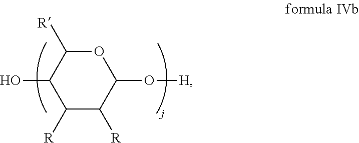
wherein each R′ is independently selected from the group consisting of CH₂OR^{a}, CH₃, CH₂F, CH₂Cl, CH₂Br, CH₂I, CH₂N₃, CH₂CN, CH₂R^{a}, CH₂N(R^{a})₂, CH₂SR^{a}, CH₂Si(R^{a})₃, CH₂CHO, CH₂COOH, CH₂COOR^{a}, CH₂CON(R^{a})₂, CH₂CHNR^{a}, OR^{a}, H, F, Cl, Br, I, N₃, CN, R^{a}, N(R^{a})₂, SR^{a}, Si(R^{a})₃, CHO, COOH, COOR^{a}, CON(R^{a})₂, and CHNR^{a},
wherein each R is independently selected from the group consisting of OR^{a}, H, F, Cl, Br, I, N₃, CN, R^{a}, N(R^{a})₂, SR^{a}, and Si(R^{a})₃,
wherein each R^{a} is independently selected from the group consisting of H, alkyl, substituted alkyl, alkenyl, substituted alkenyl, alkynyl, substituted alkynyl, acyl, substituted acyl, aryl, substituted aryl, benzyl, and substituted benzyl, and wherein j is an integer 1 or more.

18. The method according to any one of the preceding claims, wherein the second glucose-based compound has the structure of formula IVc:

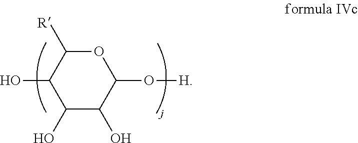

19. The method according to any one of the preceding claims, wherein the delta-cyclodextrin or the delta-cyclodextrin derivative has the structure of formula I or formula Ib, and wherein at least 1, 2, 3, 4, 5, 6, 7, 8, 9, 10, 11, 12, 13, 14, 15, 16, 17, 18, 19, 20, 21, 22, 23, 24, 25, 26, or 27 of the moieties R and/or R′ is/are selected from the group consisting of OR^{a}, H, F, Cl, Br, I, N₃, CN, R^{a}, N(R^{a})₂, SR^{a}, and Si(R^{a})₃.

20. The method according to any one of the preceding claims, wherein the delta-cyclodextrin or the delta-cyclodextrin derivative has the structure of formula I or formula Ib, and wherein at least 1, 2, 3, 4, 5, 6, 7, 8, 9, 10, 11, 12, 13, 14, 15, 16, 17, 18, 19, 20, 21, 22, 23, 24, 25, 26, or 27 of the moieties R and/or R′ is/are selected from the group consisting of OR^{a}, H, F, Cl, Br, I, N₃, CN, R^{a}, N(R^{a})₂, SR^{a}, and Si(R^{a})₃, and wherein the remaining moieties are selected from the group consisting of CH₂OR^{a}, CH₃, CH₂F, CH₂Cl, CH₂Br, CH₂I, CH₂N₃, CH₂CN, CH₂R^{a}, CH₂N(R^{a})₂, CH₂SR^{a}, CH₂Si(R^{a})₃, CH₂CHO, CH₂COOH, CH₂COOR^{a}, CH₂CON(R^{a})₂, CH₂CHNR^{a}, OR^{a}, H, F, Cl, Br, I, N₃, CN, R^{a}, N(R^{a})₂, SR^{a}, Si(R^{a})₃, CHO, COOH, COOR^{a}, CON(R^{a})₂, and CHNR^{a}.

21. The method according to any one of the preceding claims, wherein the delta-cyclodextrin or the delta-cyclodextrin derivative has the structure of formula Ia, and wherein at least 1, 2, 3, 4, 5, 6, 7, 8, or 9 of the moiety R is/are selected from the group consisting of CH₂OR^{a}, CH₃, CH₂F, CH₂Cl, CH₂Br, CH₂I, CH₂N₃, CH₂CN, CH₂R^{a}, CH₂N(R^{a})₂, CH₂SR^{a}, CH₂Si(R^{a})₃, CH₂CHO, CH₂COOH, CH₂COOR^{a}, CH₂CON(R^{a})₂, CH₂CHNR^{a}, OR^{a}, H, F, C, Br, I, N₃, CN, R^{a}, N(R^{a})₂, SR^{a}, Si(R^{a})₃, CHO, COOH, COOR^{a}, CON(R^{a})₂, and CHNR^{a}.

22. The method according to any one of the preceding claims, wherein the delta-cyclodextrin or the delta-cyclodextrin derivative has the structure of formula Ic, and wherein at least 1, 2, 3, 4, 5, 6, 7, 8, or 9 of the moiety R′ is/are selected from the group consisting of CH₂OR^{a}, CH₃, CH₂F, CH₂Cl, CH₂Br, CH₂I, CH₂N₃, CH₂CN, CH₂R^{a}, CH₂N(R^{a})₂, CH₂SR^{a}, CH₂Si(R^{a})₃, CH₂CHO, CH₂COOH, CH₂COOR^{a}, CH₂CON(R^{a})₂, CH₂CHNR^{a}, OR^{a}, H, F, Cl, Br, I, N₃, CN, R^{a}, N(R^{a})₂, SR^{a}, Si(R^{a})₃, CHO, COOH, COOR^{a}, CON(R^{a})₂, and CHNR^{a}.

23. The method according to any one of the preceding claims, wherein the delta-cyclodextrin or the delta-cyclodextrin derivative has the structure of formula Ib wherein at least 1, 2, 3, 4, 5, 6, 7, 8, 9, 10, 11, 12, 13, 14, 15, 16, 17, or 18 of the moiety R is/are selected from the group consisting of OR^{a}, H, F, Cl, Br, I, N₃, CN, R^{a}, N(R^{a})₂, SR^{a}, and Si(R^{a})₃.

24. The method according to any one of the preceding claims, wherein the delta-cyclodextrin or the delta-cyclodextrin derivative has the structure of formula Ia and wherein at least 1, 2, 3, 4, 5, 6, 7, 8, or 9 of the moiety R is/are CH₂OH.

25. The method according to any one of the preceding claims, wherein the delta-cyclodextrin or the delta-cyclodextrin derivative has the structure of formula Ib and wherein at least 1, 2, 3, 4, 5, 6, 7, 8, 9, 10, 11, 12, 13, 14, 15, 16, 17, or 18 of the moiety R is/are OH.

26. The method according to any one of the preceding claims, wherein the delta-cyclodextrin or the delta-cyclodextrin derivative has the structure of formula Ib and wherein at least 1, 2, 3, 4, 5, 6, 7, 8, or 9 of the moiety R′ is/are CH₂OH.

27. The method according to any one of the preceding claims, wherein the delta-cyclodextrin or the delta-cyclodextrin derivative has the structure of formula Ic and wherein at least 1, 2, 3, 4, 5, 6, 7, 8, or 9 of the moiety R′ is/are CH₂OH.

28. The method according to any one of the preceding claims, wherein the delta-cyclodextrin or the delta-cyclodextrin derivative has the structure of formula Ib wherein at least 1, 2, 3, 4, 5, 6, 7, 8, or 9 of the moiety R′ is/are selected from the group consisting of CH₂OR^{a}, CH₃, CH₂F, CH₂Cl, CH₂Br, CH₂I, CH₂N₃, CH₂CN, CH₂R^{a}, CH₂N(R^{a})₂, CH₂SR^{a}, CH₂Si(R^{a})₃, CH₂CHO, CH₂COOH, CH₂COOR^{a}, CH₂CON(R^{a})₂, CH₂CHNR^{a}, OR^{a}, H, F, Cl, Br, I, N₃, CN, R^{a}, N(R^{a})₂, SR^{a}, Si(R^{a})₃, CHO, COOH, COOR^{a}, CON(R^{a})₂, and CHNR^{a}.

29. The method according to any one of the preceding claims, wherein the delta-cyclodextrin or the delta-cyclodextrin derivative has the structure of formula Ic wherein at least 1, 2, 3, 4, 5, 6, 7, 8, or 9 of the moiety R′ is/are selected from the group consisting of CH₂OR^{a}, CH₃, CH₂F, CH₂Cl, CH₂Br, CH₂I, CH₂N₃, CH₂CN, CH₂R^{a}, CH₂N(R^{a})₂, CH₂SR^{a}, CH₂Si(R^{a})₃, CH₂CHO, CH₂COOH, CH₂COOR^{a}, CH₂CON(R^{a})₂, CH₂CHNR^{a}, OR^{a}, H, F, Cl, Br, I, N₃, CN, R^{a}, N(R^{a})₂, SR^{a}, Si(R^{a})₃, CHO, COOH, COOR^{a}, CON(R^{a})₂, and CHNR^{a}.

30. The method according to any one of the preceding claims, wherein the glucose-based compound is selected from the group consisting of: glucose, alpha-cyclodextrin, beta-cyclodextrin, gamma-cyclodextrin, a second cyclodextrin, a second large-ring cyclodextrin, amylose, synthetic amylose, glycogen, maltodextrin, dextrin, cycloamylose, amylopectin, starch, modified starch such as soluble starch, limit-dextrin and other alpha-glucans such as pullulan or any mixture of all of the above.

31. The method according to any one of preceding claims, wherein the glucose-based compound is glucose, a glucose derivative, a modified glucose, a glucose-based polysaccharide, a glucose-based polysaccharide derivative, a modified glucose-based polysaccharide, a second cyclodextrin, or a second cyclodextrin derivative.

32. The method according to any one of the preceding claims, wherein the second cyclodextrin is alpha-cyclodextrin, beta-cyclodextrin, or gamma-cyclodextrin.

33. The method according to any one of the preceding claims, wherein the second large-ring cyclodextrin is a cyclodextrin having a size of 10 glucose moieties or more, or a derivative of a cyclodextrin having a size of 10 glucose moieties or more.

34. The method according to any one of the preceding claims, wherein the second cyclodextrin derivative is an alpha-cyclodextrin derivative, a beta-cyclodextrin derivative, or a gamma-cyclodextrin derivative.

35. The method according to any one of the preceding claims, wherein the second cyclodextrin derivative is a modified cyclodextrin, such as a modified alpha-cyclodextrin, a modified beta-cyclodextrin, or a modified gamma-cyclodextrin.

36. The method according to any one of the preceding claims, wherein the modification is a substitution of one or more of the OH moieties present on the glucose-based compound by a moiety independently selected from the group consisting of OR^{a}, H, F, Cl, Br, I, N₃, CN, R^{a}, N(R^{a})₂, SR^{a}, Si(R^{a})₃, and/or wherein the modification is a substitution of one or more of the CH₂OH moieties present on the glucose-based compound by a moiety independently selected from the group consisting of CH₂OR^{a}, CH₃, CH₂F, CH₂Cl, CH₂Br, CH₂I, CH₂N₃, CH₂CN, CH₂R^{a}, CH₂N(R^{a})₂, CH₂SR^{a}, CH₂Si(R^{a})₃, CH₂CHO, CH₂COOH, CH₂COOR^{a}, CH₂CON(R^{a})₂, CH₂CHNR^{a}, OR^{a}, H, F, Cl, Br, I, N₃, CN, R^{a}, N(R^{a})₂, SR^{a}, Si(R^{a})₃, CHO, COOH, COOR^{a}, CON(R^{a})₂, and CHNR^{a},
wherein each R^{a} is independently selected from the group consisting of H, alkyl, substituted alkyl, alkenyl, substituted alkenyl, alkynyl, substituted alkynyl, acyl, substituted acyl, aryl, substituted aryl, benzyl, and substituted benzyl.

37. The method according to any one of the preceding claims, wherein the modified cyclodextrin derivative is a deoxy cyclodextrin, such as a monodeoxy cyclodextrin, such as a 6-deoxy cyclodextrin, such as 6-deoxy alpha-cyclodextrin, such as mono-6-deoxy alpha-cyclodextrin.

38. The method according to any one of the preceding claims, wherein n is 1, 2, 3, 4,5,6,7,8, 9, 10, 11, or 12.

39. The method according to any one of the preceding claims, wherein n is 9, 10, 11, or 12.

40. The method according to any one of the preceding claims, wherein the ion of formula [B₁₂ClₙH₁₂₋ₙ]²⁻ is selected from the group consisting of (B₁₂Cl₁₂)²⁻, (B₁₂Cl₁₁H)²⁻, (B₁₂Cl₁₀H₂)²⁻, (B₁₂Cl₉H₃)²⁻, (B₁₂Cl₈H₄)²⁻, (B₁₂Cl₇H₅)²⁻, (B₁₂Cl₆H₆)²⁻, (B₁₂Cl₅H₇)²⁻, (B₁₂Cl₄H₈)²⁻, (B₁₂Cl₃H₉)²⁻, (B₁₂Cl₂H₁₀)²⁻, and (B₁₂ClH₁₁)²⁻.

41. The method according to any one of the preceding claims, wherein the ion of formula [B₁₂ClₙH₁₂₋ₙ]²⁻ is selected from the group consisting of (B₁₂Cl₁₂)²⁻, (B₁₂Cl₁₁H)²⁻, (B₁₂Cl₁₀H₂)²⁻, and (B₁₂Cl₉H₃)²⁻.

42. The method according to any one of the preceding claims, wherein the ion of formula (B₁₂ClₙH₁₂₋ₙ)² is (B₁₂Cl₁₂)²⁻.

43. The method according to any one of the preceding claims, wherein the ion of formula (B₁₂ClₙH₁₂₋ₙ)²⁻ is from a salt of formula M₂(B₁₂ClₙH₁₂₋ₙ) or M(B₁₂ClₙH₁₂₋ₙ), wherein M is a monovalent or a divalent cation.

44. The method according to any one of the preceding claims, wherein M is selected from the group consisting of alkali metal ions such as Li⁺, Na⁺, K⁺, Cs⁺, earth-alkali metal ions such as Mg²⁺ and Ca²⁺, NH₄⁺, (RP)₄N⁺, and (RP)₄P⁺, wherein each RP is independently selected from the group consisting of H, alkyl, substituted alkyl, alkenyl, substituted alkenyl, alkynyl, substituted alkynyl, acyl, substituted acyl, aryl, substituted aryl, benzyl, and substituted benzyl.

45. The method according to any one of the preceding claims, wherein the salt of formula M₂(B₁₂ClₙH₁₂₋ₙ) is Na₂(B₁₂Cl₁₂).

46. The method according to any one of the preceding claims, wherein the cyclodextrin glucanotransferase is a cyclodextrin glucanotransferase as defined by Enzyme Commission (EC) number 2.4.1.19.

47. The method according to any one of the preceding claims, wherein the cyclodextrin glucanotransferase is an enzyme capable of catalysing the reactions catalysed by enzymes defined by Enzyme Commission (EC) number 2.4.1.19.

48. The method according to any one of the preceding claims, wherein the cyclodextrin glucanotransferase is selected from the group of enzymes defined by Enzyme Commission (EC) number 2.4.1.25.

49. The method according to any one of the preceding claims, wherein the cyclodextrin glucanotransferase is selected from the group of enzymes defined by Enzyme Commission (EC) number 3.2.1.54.

50. The method according to any one of the preceding claims, wherein the cyclodextrin glucanotransferase is CGTase, catalogue number 970390 from Amano Enzyme Europe Limited.

51. The method according to any one of the preceding claims, wherein the solvent is a protic solvent.

52. The method according to any one of the preceding claims, wherein the solvent is a polar solvent.

53. The method according to any one of the preceding claims, wherein the solvent is a protic, polar solvent.

54. The method according to any one of the preceding claims, wherein the solvent comprises two or more solvents.

55. The method according to any one of the preceding claims, wherein the solvent comprises water and an organic solvent.

56. The method according to any one of the preceding claims, wherein the solvent comprises or is dimethylformamide, dimethylsulfoxide, acetone, acetonitrile, isopropanol, ethanol, or methanol.

57. The method according to any one of the preceding claims, wherein the solvent comprises or is an aqueous solvent.

58. The method according to any one of the preceding claims, wherein the aqueous solvent comprises or is water.

59. The method according to any one of the preceding claims, wherein the solvent comprises or is an aqueous buffer.

60. The method according to any one of the preceding claims, wherein the aqueous buffer is a phosphate buffer.

61. The method according to any one of the preceding claims, wherein the concentration of the aqueous buffer is 1 to 500 mM, such as 1 to 250 mM, such as 5 to 150 mM, such as 10 to 100 mM, such as 20 to 100 mM, such as about 50 mM.

62. The method according to any one of the preceding claims, wherein the solvent has a pH of 3.5 to 11.0, such as 4.0 to 9.0, such as 4.5 to 9.0, such as 5.5 to 8.5, such as 5.5 to 8.5, such as 6.0 to 8.5, such as 6.5 to 8.5, such as 7.0 to 8.0, such as about 7.5.

63. The method according to any one of the preceding claims, wherein the solvent has a pH within the working pH range of the cyclodextrin glucanotransferase.

64. The method according to any one of the preceding claims, wherein the solvent has a pH of 4.0 to 4.5, such as 4.5 to 5.0, such as 5.0 to 5.5, such as 5.5 to 6.0, such as 6.0 to 6.5, such as 6.5 to 7.0, such as 7.0 to 7.5, such as 7.5 to 8.0, such as 8.0 to 8.5, such as 8.5 to 9.0.

65. The method according to any one of the preceding claims, wherein the incubation is carried out at 0 to 120° C., such as 0 to 100° C., such as 0 to 80° C., such as 0 to 70° C., such as 0 to 60° C., such as 5 to 45° C., 10 to 40° C., such as 15 to 40° C., such as 20 to 35° C.

66. The method according to any one of the preceding claims, wherein the incubation is carried out at 0 to 5° C., such as 5 to 10° C., such as 10 to 15° C., such as 15 to 20° C., such as 20 to 25° C., such as 25 to 30° C., such as 30 to 35° C., such as 35 to 40° C., such as 40 to 45° C.

67. The method according to any one of the preceding claims, wherein the incubation is carried out for 10 to 200 hours, such as 10 to 150 hours, such as 10 to 100 hours, such as 10 to 80 hours, such as 20 to 70 hours, such as 30 to 60 hours, such as 35 to 55 hours, such as about 40 to 50 hours.

68. The method according to any one of the preceding claims, wherein the incubation is carried out for 10 to 15 hours, such as 15 to 20 hours, such as 20 to 25 hours, such as 25 to 30 hours, such as 30 to 35 hours, such as 35 to 40, such as 40 to 45 hours, such as 45 to 50 hours, such as 50 to 55 hours, such as 55 to 60 hours, such as 60 to 65 hours, such as 65 to 70 hours, such as 70 to 75 hours, such as 75 to 80 hours, such as 80 to 85 hours, such 85 to 90 hours, such as 90 to 95 hours, such as 95 to 100 hours.

69. The method according to any one of the preceding claims, wherein the incubation is carried out for at least 24 hours, such as at least 36 hours, such as at least 42 hours, such as at least 48 hours.

70. The method according to any one of the preceding claims, wherein the incubation is carried out for about 42 hours or about 48 hours.

71. The method according to any one of the preceding claims, wherein the ratio of glucose-based compound to ion of formula (B₁₂ClₙH₁₂₋ₙ)²⁻ is between 99:1 and 20:80 (w/w), such as between 90:10 and 65:35.

72. The method according to any one of the preceding claims, wherein the ratio of glucose-based compound to ion of formula (B₁₂ClₙH₁₂₋ₙ)²⁻ is 99:1 to 98:2 (w/w), such as 98:2 to 95:5, such as 95:5 to 90:10, such as 90:10 to 85:15, such as 85:15 to 80:20, such as 80:20 to 75:25, such as 75:25 to 70:30, such as 70:30 to 65:35, such as 65:35 to 60:40, such as 60:40 to 55:45, such as 55:45 to 50:50, such as 50:50 to 45:55, such as 45:55 to 40:60, such as 40:60 to 35:65, such as 35:65 to 30:70, such as 30:70 to 25:75, such as 25:75 to 20:80.

73. The method according to any one of the preceding claims, wherein the amount of ion of formula (B₁₂ClₙH₁₂₋ₙ)²⁻ is 2 to 50 mol % per monosaccharide moiety in the glucose-based compound, such as 2 to 45 mol %, such as 3 to 40 mol %, such as 4 to 35 mol %, such as 5 to 30 mol %, such as 6 to 25 mol %.

74. The method according to any one of the preceding claims, wherein the amount of ion of formula (B₁₂ClₙH₁₂₋ₙ)²⁻ is 2 to 3 mol %, such as 3 to 4 mol %, such as 4 to 5 mol %, such as 5 to 6 mol %, such as 6 to 7 mol %, such as 7 to 8 mol %, such as 8 to 9 mol %, such as 9 to 10 mol %, such as 10 to 11 mol %, such as 11 to 12 mol %, such as 12 to 13 mol %, such as 13 to 14 mol %, such as 14 to 15 mol %, such as 15 to 17 mol %, such as 17 to 20 mol %, such as 20 to 25 mol %, such as 25 to 30 mol %, such as 30 to 35 mol %, such as 35 to 40 mol %, such as 40 to 45 mol %, such as 45 to 50 mol %.

75. The method according to any one of the preceding claims, wherein the mixture comprises an amount of cyclodextrin glucanotransferase corresponding to 0.1 to 100% (v/v) cyclodextrin glucanotransferase stock solution, such as 0.1 to 60%, such as 0.1 to 50%, such as 0.5 to 40%, such as 0.5 to 30%, such as 1.0 to 20%, such as 1 to 15%, such as 1 to 10% stock solution.

76. The method according to any one of the preceding claims, wherein the solvent is cyclodextrin glucanotransferase stock solution.

77. The method according to any one of the preceding claims, wherein the mixture comprises an amount of cyclodextrin glucanotransferase corresponding to 0.1 to 0.2% (v/v) cyclodextrin glucanotransferase stock solution, such as 0.2 to 0.3%, such as 0.3 to 0.4%, such as 0.4 to 0.5%, such as 0.5 to 0.6%, such as 0.6 to 0.7%, such as 0.7 to 0.8%, such as 0.8 to 0.9%, such as 0.9 to 1.0%, such as 1.0 to 1.5%, such as 1.5 to 2.0%, such as 2.0 to 2.5%, such as 2.5 to 3.0%, such as 3.0 to 4.0%, such as 4.0 to 5.0%, such as 5.0 to 6.0%, such as 6.0 to 7.0%, such as 7.0 to 8.0%, such as 8.0 to 9.0%, such as 9.0 to 10%, such as 10 to 15%, such as 15 to 20%, such as 20 to 25%, such as 25 to 30%, such as 30 to 40%, such as 40 to 50% cyclodextrin glucanotransferase stock solution.

78. The method according to any one of the preceding claims, wherein the cyclodextrin glucanotransferase stock solution corresponds to or is cyclodextrin glucanotransferase having catalogue no. 970390 from Amano Enzyme Europe Limited.

79. The method according to any one of the preceding claims, wherein the concentration of the glucose-based compound in the solvent is 0.1 to 200 g/L, such as 0.1 to 150 g/L, such as 0.1 to 100 g/L, such as 0.2 to 90 g/L, such as 0.5 to 80 g/L, such as 0.5 to 70 g/L, such as 1 to 60 g/L, such as 1 to 50 g/L, such as 1 to 50 g/L, such as 1 to 40 g/L, such as 1 to 30 g/L, such as 1 to 25 g/L.

80. The method according to any one of the preceding claims, wherein the concentration of the glucose-based compound in the solvent is 0.1 to 0.2 g/L, such as 0.2 to 0.5 g/L, such as 0.5 to 1 g/L, such as 1 to 2 g/L, such as 2 to 5 g/L, such as 5 to 10 g/L, such as 10 to 15 g/L, such as 15 to 20 g/L, such as 20 to 25 g/L, such as 25 to 30 g/L, such as 30 to 40 g/L, such as 40 to 50 g/L, such as 50 to 60 g/L, such as 60 to 70 g/L, such as 70 to 80 g/L, such as 80 to 90 g/L, such as 90 to 100 g/L.

81. The method according to any one of the preceding claims, wherein the concentration of the ion of formula (B₁₂ClₙH₁₂₋ₙ)²⁻ in the solvent is 0.1 to 300 mM, such a 0.1 to 250 mM, such as 1 to 200 mM, such as 1 to 150 mM, such as 1 to 100 mM, such as 0.2 to 90 mM, such as 0.3 to 80 mM, such as 0.4 to 70 mM, such as 0.5 to 60 mM, such a 0.6 to 50 mM, such as 0.7 to 40 mM, such as 0.8 to 30 mM, such as 0.9 to 25 mM, such as 1.0 to 20 mM, such as 1.0 to 15 mM, such as 1.0 to 10 mM.

82. The method according to any one of the preceding claims, wherein the concentration of the ion of formula (B₁₂ClₙH₁₂₋ₙ)²⁻ in the solvent is 0.1 to 0.2 mM such as 0.2 to 0.3 mM, such as 0.3 to 0.4 mM, such as 0.4 to 0.5 mM, such as 0.5 to 0.6 mM, such as 0.6 to 0.7 mM, such as 0.7 to 0.8 mM, such as 0.8 to 0.9 mM, such as 0.9 to 1.0 mM, such as 1.0 to 1.5 mM, such as 1.5 to 2.0 mM, such as 2 to 3 mM, such as 3 to 4 mM, such as 4 to 5 mM, such as 5 to 6 mM such as 6 to 7 mM, such as 7 to 8 mM, such as 8 to 9 mM, such as 9 to 10 mM, such as 10 to 15 mM, such as 15 to 20 mM, such as 20 to 25 mM, such as 25 to 30 mM, such as 30 to 40 mM, such as 40 to 50 mM, such as 50 to 60 mM, such as 60 to 70 mM, such as 70 to 80 mM, such as 80 to 90 mM, such as 90 to 100 mM.

83. The method according to any one of the preceding claims, further comprising a step of chemically modifying the thereby obtained delta-cyclodextrin or delta-cyclodextrin derivative.

84. The method according to any one of the preceding claims, wherein:
a. the incubation is carried out for 30 to 60 hours, and
b. the incubation is carried out at 0 to 45° C.

85. The method according to any one of the preceding claims, wherein:
a. the incubation is carried out for 40 to 56 hours, and
b. the incubation is carried out at 20 to 30° C.

86. The method according to any one of the preceding claims, wherein:
a. the glucose-based compound is starch, a cyclodextrin such as alpha-cyclodextrin, or a modified cyclodextrin such as mono-6-deoxy alpha-cyclodextrin; and
b. the ion of formula (B₁₂ClₙH₁₂₋ₙ)² is (B₁₂Cl₁₂)²⁻, (B₁₂Cl₁₁H)²⁻,(B₁₂Cl₁₀H₂)²⁻, or (B₁₂Cl₉H₃)²⁻.

87. The method according to any one of the preceding claims, wherein:
a. the concentration of the glucose-based compound in the solvent is 1 to 50 g/L,
b. the concentration of the ion of formula (B₁₂ClₙH₁₂₋ₙ)²⁻ in the solvent is 0.5 to 25 mM, and
c. the mixture comprises an amount of cyclodextrin glucanotransferase corresponding to 0.1 to 50% (v/v) cyclodextrin glucanotransferase stock solution as set out in any one of the preceding claims.

88. The method according to any one of the preceding claims, wherein:
a. the concentration of the glucose-based compound in the solvent is 5 to 25 g/L,
b. the concentration of the ion of formula (B₁₂ClₙH₁₂₋ₙ)²⁻ in the solvent is 0.5 to 10 mM, and
c. the mixture comprises an amount of cyclodextrin glucanotransferase corresponding to 1 to 10% (v/v) cyclodextrin glucanotransferase stock solution as set out in any one of the preceding claims.

89. The method according to any one of the preceding claims, wherein:
a. the incubation is carried out for 30 to 60 hours,
b. the incubation is carried out at 0 to 45° C.
c. the concentration of the glucose-based compound in the solvent is 1 to 50 g/L,
d. the concentration of the ion of formula (B₁₂ClₙH₁₂₋ₙ)²⁻ in the solvent is 0.5 to 25 mM, and
e. the mixture comprises an amount of cyclodextrin glucanotransferase corresponding to 0.1 to 50% (v/v) cyclodextrin glucanotransferase stock solution as set out in any one of the preceding claims.

90. The method according to any one of the preceding claims, wherein:
a. the incubation is carried out for 40 to 56 hours,
b. the incubation is carried out at 20 to 30° C.
c. the concentration of the glucose-based compound in the solvent is 5 to 25 g/L,
d. the concentration of the ion of formula (B₁₂ClₙH₁₂₋ₙ)²⁻ in the solvent is 0.5 to 10 mM, and
e. the mixture comprises an amount of cyclodextrin glucanotransferase corresponding to 1 to 10% (v/v) cyclodextrin glucanotransferase stock solution.

91. The method according to any one of the preceding claims, wherein:
a. the incubation is carried out for 30 to 60 hours,
b. the incubation is carried out at 0 to 45° C.
c. the concentration of the glucose-based compound in the solvent is 1 to 50 g/L,
d. the concentration of the ion of formula (B₁₂ClₙH₁₂₋ₙ)²⁻ in the solvent is 0.5 to 25 mM,
e. the mixture comprises an amount of cyclodextrin glucanotransferase corresponding to 0.1 to 50% (v/v) cyclodextrin glucanotransferase stock solution as set out in any one of the preceding claims,
f. the glucose-based compound is selected from glucose, alpha-cyclodextrin, beta-cyclodextrin, gamma-cyclodextrin, a modified alpha-cyclodextrin, a modified beta-cyclodextrin, a modified gamma-cyclodextrin, starch such as soluble starch, and a glucan, and
g. the ion of formula [B₁₂ClₙH₁₂₋ₙ]²⁻ is selected from the group consisting of (B₁₂Cl₁₂)²⁻, (B₁₂Cl₁₁H)²⁻, (B₁₂Cl₁₀H₂)²⁻, (B₁₂Cl₉H₃)²⁻, (B₁₂Cl₈H₄)²⁻, (B₁₂Cl₇H₅)²⁻, (B₁₂Cl₆H₆)²⁻, (B₁₂Cl₅H₇)²⁻, (B₁₂Cl₄H₈)²⁻, (B₁₂Cl₃H₉)²⁻, (B₁₂Cl₂H₁₀)²⁻, and (B₁₂ClH₁₁)²⁻.

92. The method according to any one of the preceding claims, wherein:
a. the incubation is carried out for 40 to 56 hours,
b. the incubation is carried out at 20 to 30° C.
c. the concentration of the glucose-based compound in the solvent is 5 to 25 g/L,
d. the concentration of the ion of formula (B₁₂ClₙH₁₂₋ₙ)²⁻ in the solvent is 0.5 to 10 mM,
e. the mixture comprises an amount of cyclodextrin glucanotransferase corresponding to 1 to 10% (v/v) cyclodextrin glucanotransferase stock solution.
f. the glucose-based compound is selected from glucose, alpha-cyclodextrin, beta-cyclodextrin, gamma-cyclodextrin, a modified alpha-cyclodextrin, a modified beta-cyclodextrin, a modified gamma-cyclodextrin, starch such as soluble starch, and a glucan, and
g. the ion of formula [B₁₂ClₙH₁₂₋ₙ]²⁻ is selected from the group consisting of (B₁₂Cl₁₂)₂, (B₁₂Cl₁₁H)²⁻, (B₁₂Cl₁₀H₂)²⁻, and (B₁₂Cl₉H₃)²⁻.

93. The method according to any one of the preceding claims, said method further comprising a step of heating the mixture to at least 70° C., such as at least 75° C., such as at least 80° C., such as at least 85° C., such as at least 90° C., such as at least 95° C., such as at least 100° C., such as at least 110° C., such as at least 120° C., such as at least 130° C.

94. The method according to any one of the preceding claims, wherein said heating the mixture is for 1 to 60 min, such as 5 to 50 min, such as 5 to 40 min, such as 5 to 30 min, such as 5 to 20 min, such as for about 15 min.

95. The method according to any one of the preceding claims, wherein said heating the mixture is for at least 5 min, such as at least 10 min, such as at least 15 min.

96. The method according to any one of the preceding claims, said heating step being carried out subsequently to the incubation step.

97. The method according to any one of the preceding claims, said method further comprising a step of partially evaporating the solvent from mixture.

98. The method according to any one of the preceding claims, wherein said evaporating the mixture comprises removing 10 to 20% of the solvent volume, such as 20 to 30%, such as 30 to 40%, such as 40 to 50%, such as 50 to 60%, such as 60 to 70%, such as 70 to 80%, such as 80 to 90%.

99. The method according to any one of the preceding claims, wherein said partially evaporating the solvent from the mixture is carried out under reduced pressure and/or by heating the mixture.

100. The method according to any one of the preceding claims, wherein said partially evaporating the solvent from the mixture is carried out at atmospheric pressure and/or without heating the mixture.

101. The method according to any one of the preceding claims, wherein said partially evaporating the solvent from the mixture is effective in precipitating the cyclodextrin glucanotransferase.

102. The method according to any one of the preceding claims, wherein the mixture comprises a solid phase comprising cyclodextrin glucanotransferase and a liquid phase comprising delta-cyclodextrin or a delta-cyclodextrin derivative, and wherein said method further comprising a step of separating the solid phase and the liquid phase of the mixture.

103. The method according to any one of the preceding claims, said step of separating the solid phase comprising cyclodextrin glucanotransferase and a liquid phase comprising delta-cyclodextrin or a delta-cyclodextrin derivative is carried out using centrifuging, filtering, and/or decanting.

104. The method according to any one of the preceding claims, said method further comprising a step of precipitating the delta-cyclodextrin or the delta-cyclodextrin derivative from the liquid phase.

105. The method according to any one of the preceding claims, wherein the precipitation of the delta-cyclodextrin or the delta-cyclodextrin derivative comprises adding an antisolvent to the liquid phase, whereby the delta-cyclodextrin or the delta-cyclodextrin derivative precipitates.

106. The method according to any one of the preceding claims, wherein the antisolvent is a protic solvent or an aprotic polar solvent.

107. The method according to any one of the preceding claims, wherein the antisolvent is selected from the group consisting of acetone, ethanol, isopropanol, and acetonitrile.

108. The method according to any one of the preceding claims, said method further comprising a step of filtering the precipitated delta-cyclodextrin or delta-cyclodextrin derivative.

109. The method according to any one of the preceding claims, wherein the method further comprises a step of:
a. dissolving in water and/or making a slurry in water of the delta-cyclodextrin or delta-cyclodextrin derivative,
b. adding an antisolvent, such as a protic solvent or an aprotic polar solvent, such as acetone, ethanol, isopropanol, or acetonitrile, thereby precipitating the delta-cyclodextrin or delta-cyclodextrin derivative, and
c. filtering the precipitated delta-cyclodextrin or delta-cyclodextrin derivative.

110. The method according to any one of the preceding claims, wherein the steps are carried out in the order:
a. mixing:
i. a glucose-based compound,
ii. a cyclodextrin glucanotransferase,
iii. an ion of formula (B₁₂ClₙH₁₂₋ₙ)²⁻, wherein n is 1 to 12, and
iv. a solvent to form a mixture;
b. incubating the mixture by heating;
c. partially evaporating the solvent from mixture to form a solid phase and a liquid phase;
d. separating the solid phase and the liquid phase of the mixture;
e. adding antisolvent to said liquid phase to precipitate the delta-cyclodextrin or the delta-cyclodextrin derivative; and
f. filtering the precipitated delta-cyclodextrin or the delta-cyclodextrin derivative.

111. The method according to any one of the preceding claims, further comprising recovering the ion of formula (B₁₂ClₙH₁₂₋ₙ)²⁻, said recovery comprising:
a. obtaining the supernatant from the precipitation step of the delta-cyclodextrin or the delta-cyclodextrin derivative,
b. evaporating antisolvent under reduced pressure,
c. adding hydrochloric acid, such as 37% hydrochloric acid to obtain a pH between 1 and 4, such as between 1.5 and 2.5,
d. adding a base, such as an amine base, such as an alkyl amine base, such as a trialkyl amine compound, such as triethylamine to precipitate the ion of formula (B₁₂ClₙH₁₂₋ₙ)²⁻ as a salt of said base, and
e. filtering the precipitate,
thereby obtaining the ion of formula (B₁₂ClₙH₁₂₋ₙ)²⁻ as a salt of said base.

112. A method for producing delta-cyclodextrin or a delta-cyclodextrin derivative, said method comprising in order the steps of:
a. mixing:
i. a glucose-based compound,
ii. a cyclodextrin glucanotransferase,
iii. an ion of formula (B₁₂ClₙH₁₂₋ₙ)²⁻, wherein n is 1 to 12, and
iv. a solvent
to form a mixture;
b. incubating the mixture by heating it to at least 70° C. for at least 5 min;
c. partially evaporating the solvent from mixture, whereby the cyclodextrin glucanotransferase is precipitated, thereby forming a solid phase and a liquid phase;
d. separating the solid phase and the liquid phase of the mixture:
e. adding antisolvent such as a protic solvent or an aprotic polar solvent to said liquid phase, whereby the delta-cyclodextrin or the delta-cyclodextrin derivative precipitates; and
f. filtering the precipitated delta-cyclodextrin or delta-cyclodextrin derivative;
thereby obtaining the delta-cyclodextrin or the delta-cyclodextrin derivative.

113. A delta-cyclodextrin or a delta-cyclodextrin derivative obtained from the method according to any one of the preceding claims.

114. A composition comprising the delta-cyclodextrin or the delta-cyclodextrin derivative obtained according to any one of the preceding claims and an ion of formula (B₁₂ClₙH₁₂n)²⁻.

115. A composition comprising:
a. a glucose-based compound according to any one of the preceding claims,
b. a cyclodextrin glucanotransferase according to any one of the preceding claims, and
c. an ion of formula (B₁₂ClₙH₁₂₋ₙ)²⁻, wherein n is 1 to 12, according to any one of the preceding claims.

116. A kit of parts comprising:
a. a glucose-based compound according to any one of the preceding claims,
b. a cyclodextrin glucanotransferase according to any one of the preceding claims, and
c. a salt comprising an ion of formula (B₁₂ClₙH₁₂₋ₙ)²⁻, wherein n is 1 to 12, according to any one of the preceding claims.
